(19) United States
(12) Patent Application Publication
(10) Pub. No.: US 2005/0208031 A1
(43) Pub. Date: Sep. 22, 2005

(54) Inhibition of calcium-independent phospholipases A2beta or A2gamma inhibit hormone-induced differentiation of 3T3-L1 preadipocytes

(76) Inventor: Richard W. Gross, Chesterfield, MO (US)

Correspondence Address: Patrick W. Rasche;Armstrong Teasdale LLP, One Metropolitan Square, Ste 2600, St. Louis, MO, 63102-2740, US

(21) Appl. No.: 11/022,579

(22) Filed: Dec. 23, 2004

Related U.S. Application Data
(60) Provisional application No. 60/532,536, filed on Dec. 24, 2003.

(51) Int. Cl. A61K 48/00; C12N 15/85

(52) U.S. Cl. 424/93.21; 514/44; 435/455

(57) ABSTRACT

A method for identifying an agonist exhibiting molecular or pharmacologic inhibition which is effective against the activity of at least one of iPLA₂β and iPLA₂γ which comprises culturing 3T3-L1 cells and transfecting them with negative control siRNA, siRNA directed against iPLA₂β or siring directed against iPLA₂γ prior to induction or during to differentiation or pharmacologic inhibition and observing for whether that down regulation of iPLA₂β or iPLA₂γ inhibits adipocyte differentiation.

### CROSS REFERENCE TO RELATED APPLICATIONS

This application claims the benefit of U.S. provisional patent application 60/532,536 filed Dec. 24, 2003 the contents of which are incorporated herein by reference in their entirety.

This research was supported by NIH Grant 5P01H57278-08. The government has certain rights in the invention.

### FIELD OF THE INVENTION

This invention relates to functional calcium-independent phospholipases A₂β and A₂γ and more particularly to research tools therefore and for methods of therapeutically intentionally controlling obesity in living mammals.

### BACKGROUND OF THE INVENTION

Recently, there has been an unfortunate and undesired dramatic increase in the incidence of obesity in humans in industrialized and newly developed countries⁽¹⁾. Estimates of persons who have a weight problem range from about 10-25% of teenagers and 20-50% of adults. Obesity which is regarded as a disease in some situations is characterized by the accumulation of fat tissue and at times this is referred to as body fat content.

Obesity is usually defined as a body fat content greater than 25% of the total weight for males, or greater than 30% of the total weight for females. Regardless of the cause of obesity whether it is one or more of consuming too much food energy, little exercise, genetics, low body metabolism, social and economic and psychological and emotion factors, it is an ever present problem for Americans in particular.

In humans, obesity is usually defined as a body fat content greater than 25% of the total weight for males, or greater than 30% of the total weight for females. Regardless of the cause of obesity, obesity is an ever present problem for Americans. But a fat content >18% for males and >22% for females can have untold consequences secondary to several mechanisms and disorders of metabolic function. For example, obesity can have a significant adverse impact on health care costs and provoke a higher risk of numerous illnesses, including heart attacks, strokes and diabetes.

Without being bound by theory, it is believed that obesity in humans results from an abnormal increase in white adipose tissue mass that occurs due to an increased number of adipocytes (hyperplasia) or from increased lipid mass accumulating in existing adipocytes. Obesity and the associated type 2 metabolic syndrome along with its clinical sequelae are among the major and the most rapidly increasing medical problems in America. However, to date, a lack of suitable adipocyte specific protein targets has unfortunately hampered progress in the development of effective therapeutic agents to combat the clinical sequelae of obesity.

Abnormal increases in white adipose tissue (WAT) mass leading to alterations in whole organism energy storage and utilization occur during obesity⁽²⁻⁴⁾Increased adipose tissue mass can result from either an increase in individual adipocyte cell size (hypertrophy) or from an increase in total adipocyte number (hyperplasia). Alterations in whole organism lipid homeostasis leading to increased adipocyte tissue mass are highly correlated with the metabolic syndrome accompanied by its lethal sequelae of diabetes, hypertension and atherosclerosis⁽²⁻⁵⁾. During the last decade, substantial progress has been made in understanding the biochemical events leading to adipocyte differentiation utilizing the hormone-induced 3T3-L1 cell model of adipocyte differentiation⁽⁶⁻⁹⁾. Central to this understanding has been the detailed characterization of the temporally coordinated changes in the expression of specific genes which collectively define the adipocyte phenotype. Differentiation of adipocytes is accomplished by the programmed activation of transcriptional regulatory proteins which modulate the temporally coordinated expression of mRNA and proteins which effectively reprogram 3T3-L1 cell lipid metabolism to that of a mature adipocyte. Such alterations include increased de novo fatty acid synthesis, accumulation of perilipin-coated triglyceride droplets, and the generation of lipid second messengers including eicosanoids and lysophosphatidic acid which serve as potent and specific regulators of coordinated adipocyte differentiation programs^{(3,7,10-14)}.

Phospholipases A₂ (PLA₂s) catalyze the hydrolysis of the sn-2 fatty acid substituents from glycerophospholipid substrates to yield free fatty acid (e.g. arachidonic acid) and lysophospholipid⁽¹⁵⁻⁷⁾. Mammalian phospholipases A₂ have been categorized into several classes based on their requirement for calcium ion in in vitro activity assays (i.e., millimolar, nanomolar, or no calcium requirement) leading to their broad classification into three classes of enzymes (sPLA₂, cPLA₂ and iPLA₂)⁽¹⁸⁾. Prior studies have demonstrated that eicosanoids are potent modulators of adipocyte differentiation underscoring the roles of PGE₂ and PGI₂ in inducing transformation of progenitor cells into mature adipocytes^{(19,20)}. In contrast, PGF₂α inhibits hormone-induced differentiation of 3T3-L1 cells into mature adipocytes⁽²¹⁾. In most mammalian cells, the rate-determining step in the production of biologically active eicosanoids is the release of arachidonic acid from the sn-2 position of glycerophospholipids. Despite the known importance of eicosanoids in modulating adipocyte differentiation, there is a paucity of information on the molecular identity of the specific types of intracellular phospholipases A₂ present in adipocytes, alterations in the mass and activity levels of the different intracellular phospholipase A₂ classes and types during the differentiation process and the importance of each specific type of phospholipase A₂ in adipocyte differentiation⁽¹⁴⁾.

Recent studies have demonstrated that LPA serves a dual function in adipocyte differentiation acting both as an extracellular ligand for EDG receptors^{(22,23)} and as the endogeneous intracellular ligand for the adipocyte transcriptional regulator PPARγ⁽²⁴⁾. According to current dogma, LPA produced during adipocyte differentiation results from the sequential hydrolysis of phosphatidylcholine to LPC by endogenous phospholipases A₂ and the subsequent extracellular hydrolysis of LPC to LPA catalyzed by a secreted lysophospholipase D, autotaxin⁽²²⁾. However, there is no information presently available on the types of phospholipases A₂ present in the adipocyte which contribute to eicosanoid and lysolipid production in the adipocyte.

Despite existing knowledge of the critical role of phospholipases in adipocyte signaling, enhanced clinical methodology, research tools and research methods are highly needed to identify drugs useful to treat obesity and over-weightness. It is highly desired to have new technology based on modulating the amounts of activities of the specific types of phospholipases present in the adipocyte or their mechanisms of regulation and to be able to determine their natural substrates and roles in anabolic lipid metabolism, catabolic lipid metabolism or both (e.g. triglyceride cycling).

Additionally, a screening method and research tool is needed to identify useful drugs which can be used to reduce the fat level of a living mammal and/or to maintain the fat level at a intentionally predetermined level.

### BRIEF DESCRIPTION OF THE INVENTION

In an aspect, a method for identifying an agonist exhibiting molecular biologic inhibition effective against the activity of at least one of the iPLA₂β and iPLA₂γ isoforms comprises culturing 3T3-L1 cells, transfecting the cells with nM negative control, siRNA directed against iPLA₂β isoforms, or siRNA directed against iPLA₂γ isoforms or other pharmacologic agents prior to induction to differentiation and optionally observing for whether down regulation of iPLA₂β or iPLA₂γ occurred which inhibited adipocyte differentiation. In an aspect, the agonist or inhibitor is identified and determined to be an agonist or inhibitor if protein metabolism and/or lipid metabolism is altered in a way similar or substantially similar to that of hormone induced differentiation. In an aspect, the negative control siRNA is 20 nM negative control siRNA.

In an aspect, an antagonist is identified or determined to be an antagonist if protein or lipid metabolism is altered in a way similar to that which attenuates hormone induced differentiation of 3T3-L1 cells.

In an aspect, a method for controlling the rate of differentiation of preadipocytes to adipocytes in a living mammal having differentiable preadipocytes comprises transfecting cells with siRNA directed against at least one of iPLA₂β or iPLA₂γ or the moieties (R)BEL, (S)BEL or a racemic mixture thereof.

In an aspect, a method of modulating the amount of fat in a living mammal having differentiable preadipocytes comprises treating 3T3-L1 cultured cells with 20 nM siRNA directed against iPLA₂β or siRNA directed against iPLA₂γ prior to induction of adipocyte differentiation and measuring standard indices of differentiation. In an aspect, modulation comprises reducing, effecting or retarding the amount of differentiation.

In an aspect, a method of intentionally noninvasively controlling the rate of differentiation of preadipocytes to adipocytes in a living mammal having differentiable preadipocytes and expressible iPLA₂β or iPLA₂γ comprises administering a pharmacologically effective amount of an inhibitor selected from at least one of S-BEL, R-BEL and a racemic mixture thereof and measuring the activity of iPLA₂β or iPLA₂γ in conjunction with other bio markers characterizing adipocyte fat.

In an aspect, a negative control siRNA comprises siRNA which is not directed against iPLA₂β or iPLA₂γ.

In an aspect, a method for identifying a molecular biologic inhibition by siRNA knockdown comprises transfecting 3T3-L1 cultured cells with 20 nM negative control siRNA or siRNA directed against of iPLA₂β or siRNA directed against iPLA₂γ prior to induction of preadipocytes to differentiation, growing the cultured cells in the presence of selected hormone and nutrient conditions and measuring alterations in the lipidome, comparing the flux of dynamic lipid alterations with mass spectroscopy to controls and determining that the siRNA knockdown or other drug was effective and presented inhibition if the flux of lipid metabolism was changed or adipocyte differentiation programs were altered.

In an aspect, a method of intentionally reducing or controlling fat in a living animal and/or a sample thereof comprises administering a pharmacologically effective amount of S-BEL, R-BEL or a racemic mixture thereof to the living animal whereby the amount of fat is reduced or controlled.

In an aspect, a method of screening a library for inhibitors of iPLA₂β or iPLA₂γ, determining if a drug is an inhibitor and testing those inhibitors to determine if altered fat content in mammals is presented and if the fat content is altered or substantially altered determining that the drug is an inhibitor.

In an aspect, an functional animal model useful for identifying a molecular biologic inhibition by siRNA knockdown having transfected 3T3-L1 cultured cells with 20 nM negative control siRNA, siRNA directed against of iPLA₂β or siRNA directed against iPLA₂γ prior to induction of preadipocytes to differentiation and having an environment beneficially effective to elicit differentiation to adipocytes and having effective means to measure the protein profile by comparing the amount of reacting cell protein with differentiated fat cell protein and determining that the siRNA knockdown was effective if alterations in the proteome in differentiating adult fat cells were presented.

In an aspect, a method for identifying a pharmacological inhibitor of fat in a living tissue comprises administering an effective amount of a compound to a living tissue having differentiable preadipocytes and expressible iPLA₂β or iPLA₂γ, measuring the change in activity of the expressible iPLA₂β or iPLA₂γ, and determining that the compound is a pharmacological inhibitor of fat accumulation when at least one of the net mass of fat is altered, recycling time of lipids is changed or uptake of lipids into adipocytes or efflux of lipids out of adipocytes are altered. In an aspect, a functional effect resulting from the administration of the compound is determined and identified such as an effect on fat content or hormone stimulated lipid hydrolysis from fat cells.

In an aspect, a method for identifying an agonist exhibiting molecular biologic inhibition effective against the activity of at least one of iPLA₂β or iPLA₂γ isoforms comprises culturing 3T3-L1 cells, transfecting those cells with 20 nM negative control siRNA, siRNA directed against iPLA₂β, or siRNA directed against iPLA₂γ prior to induction to differentiation, observing for down regulation of iPLA₂β or iPLA_{2γ} and inhibition of adipocyte differentiation and making a determination or identification of lipid or protein content, lipid or protein turnover, or cell proliferation based on that down regulation and inhibition thereby identifying the Agonist.

In an aspect, a screening tool and method comprises representative living tissue having differentiable preadipocytes and expressible iPLA₂β or iPLA₂γ, and an administration method of administering a silencing gene thereto or a pharmacological inhibitor effecting thereto and means for determining any change in metabolics of the tissue. In an aspect, the means comprises a representative functional biological sample. In an aspect, the sample is analyzed by ESI/MS for lipid content.

In an aspect, a method of characterizing the importance of iPLA₂β or iPLA₂γ in fat cell biology by using the temporally coordinated regulated activation or inhibition of these enzymes after an effective stimulus (meal or hormones) to modulate the medical sequelae of the metabolic syndrome including diabetes, atherosclerosis, obesity or hypertension.

A method of directing a silencing gene as a directed genomic projectile to a mRNA target comprising functional iPLA₂β and/or iPLA₂γ in a living animal to control the fat content of that living animal comprises directing siRNA against iPLA₂β or siRNA against iPLA₂γ. In an aspect, the genomic target comprises mRNA of iPLA₂β and/or iPLA₂γ. In an aspect, the functional genomic projectile siRNA is directed against mRNA encoding iPLA₂β or iPLA₂γ and reacts with mRNA encoding iPLA₂β and/or iPLA₂γ.

In an aspect, a method of intentionally controlling fat in a living animal comprising directing a recombinant projectile against an mRNA receptive target in the living animal wherein the projectile comprises siRNA directed against functional iPLA₂β or iPLA₂γ. In an aspect, the projectile is prepared outside the animal and projected inside the animal.

In an aspect, the identification of a pharmacological drug to control fat is identified by administering a drug to a living animal or a representative sample thereof and determining whether the drug inhibited the expression of iPLA₂β and/or iPLA₂γ by selective reaction therewith or reaction(s) or interaction(s) with its regulatory network so as to control or modulate fat.

In an aspect, the directing comprises an effective administration of a silencing siRNA having genomic (mRNA) targeting selectivity to iPLA₂β and/or iPLA₂γ.

A method of controlling fat in a living animal which comprises directing a recombinant projectile to and impacting a genomic target wherein the projectile comprises negative control siRNA directed against mRNA encoding iPLA₂β and/or siRNA directed against mRNA encoding iPLA₂γ.

In an aspect, a drug or siRNA is administered to an animal model to determine if the drug or siRNA is an inhibitor of the expression and/or activity of iPLA₂β or iPLA₂γ, the effect of such addition, if any, is determined and the drug or siRNA is determined to be an inhibitor based on an analysis (such as TAG content) of the tissue of living animal model.

In an aspect, a method of targeting a genomic target to control the fat content of a living animal which comprises directing siRNA directed against iPLA₂β or siRNA directed against mRNA functionally encoding iPLA₂γ encoded messages. In an aspect, the genomic target is specified through the mRNAs encoding iPLA₂β and/or iPLA₂γ.

In an aspect, an animal model which provides at least one of a genomic target and a pharmacological target respectively comprises a functional genome expressing iPLA₂β and iPLA₂γ for reactive reception to at least one of a projectile comprising siRNA or from a pharmacological drug administered to the animal model. In an aspect, the animal model is a living tissue representative of a living animal or a sample of a living animal such as tissue. In an aspect, the pharmacological drug is S-BEL, R-BEL or a racemic mixture thereof.

### BRIEF DESCRIPTION OF THE DRAWINGS

FIGS. 1-10 present data relating to this discovery.

FIG. 1 shows messenger RNA levels of cPLA₂α, iPLA₂β and iPLA₂γ in 3T3-L1 cells during differentiation.

FIG. 2 shows western blots of cPLA₂α, iPLA₂β and iPLA₂γproteins in 3T3-L1 cells during differentiation.

FIG. 3 show activities of iPLA₂ in 3T3-L1 cells during differentiation and their inhibition by BELs.

FIG. 4 shows effects of siRNAs directed against iPLA₂β or iPLA₂γ on the expression of several adipocyte markers.

FIG. 5 shows effects of siRNAs directed against iPLA₂β or iPLA₂γ on TAG accumulation during 3T3-L1 cell differentiation.

FIG. 6 shows effects of BELs on TAG accumulation during hormone-induced differentiation of 3T3-L1 cells.

FIG. 7 shows effects of siRNAs directed against iPLA₂β or iPLA₂γ on the expression of several transcription factors.

FIG. 8 shows effects of siRNAs directed against iPLA₂β or iPLA₂γ on mitotic clonal expansion.

FIG. 9 shows troglitazone rescues the expression of C/EBPα and PPARγ during the differentiation of 3T3-L1 cells pretreated with siRNAs directed against iPLA₂β or iPLA₂γ.

FIG. 10 shows up-regulation of iPLA₂β and iPLA₂γ in obese Zucker (fa/fa) rat White Adipose Tissue.

### DETAILED DESCRIPTION OF THE DRAWINGS

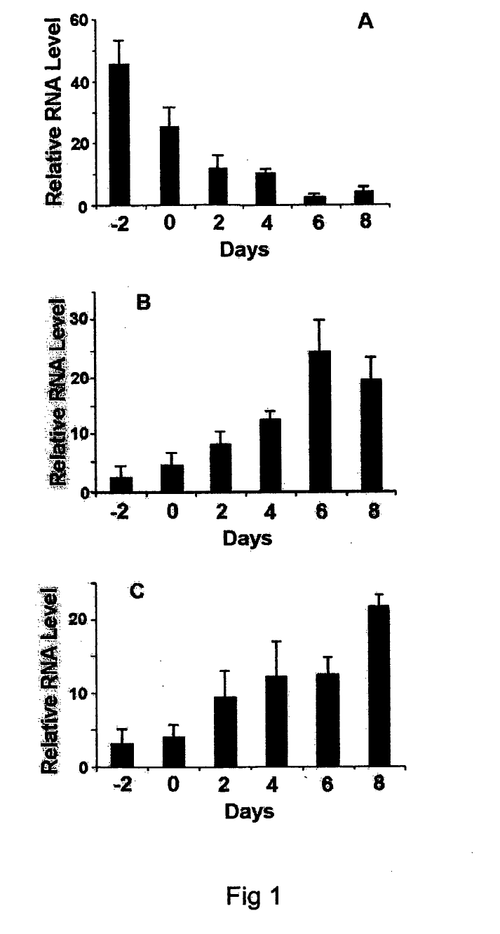

FIG. 1. Messenger RNA levels of cPLA₂α(A), iPLA₂β(B) and iPLA₂γ(C) in 3T3-L1 cells during differentiation. 3T3-L1 cells were cultured and induced to differentiate as described in “Test Procedures” hereinafter more particularly described. At indicated differentiation stages, total RNA was prepared as described in “Test Procedures.” Quantitative PCR was performed with TaqMan® PCR reagent kits in the ABI PRISM 7700 detection system utilizing GAPDH as the internal standard.

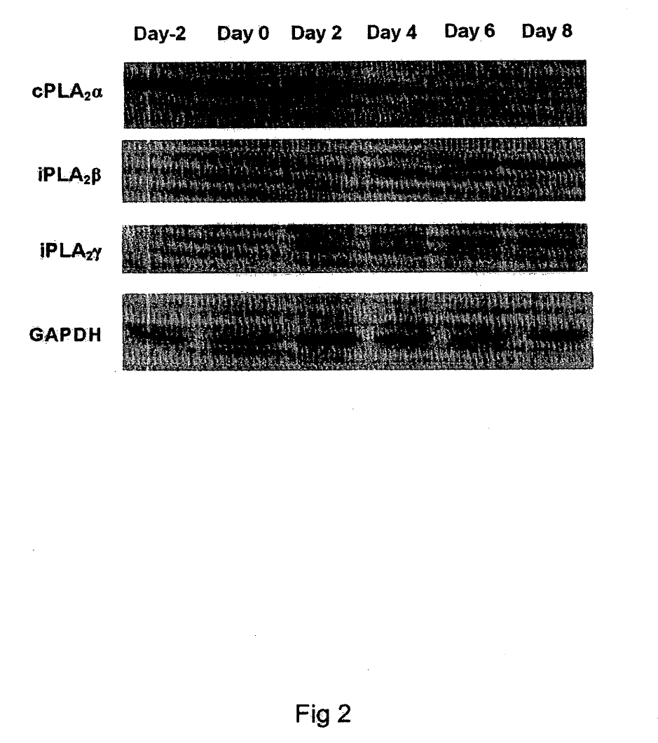

FIG. 2. Western blots of cPLA₂α, iPLA₂β and iPLA₂γ proteins in 3T3-L1 cells during differentiation. 3T3-L1 cells were cultured and induced to differentiate as described in “Test Procedures.” At indicated differentiation stages, total proteins were extracted as described in “Test Procedures.” 40 μg of proteins were loaded to each lane, separated by SDS-PAGE and transferred to Immobilon-P membranes. Powdered milk (5% (w/v)) was used to block nonspecific binding sites prior to incubation with primary antibody directed against each specific protein as indicated. After incubation with horseradish peroxidase-conjugated secondary antibody, proteins were visualized by enhanced chemiluminescence according to the instructions of the manufacturer.

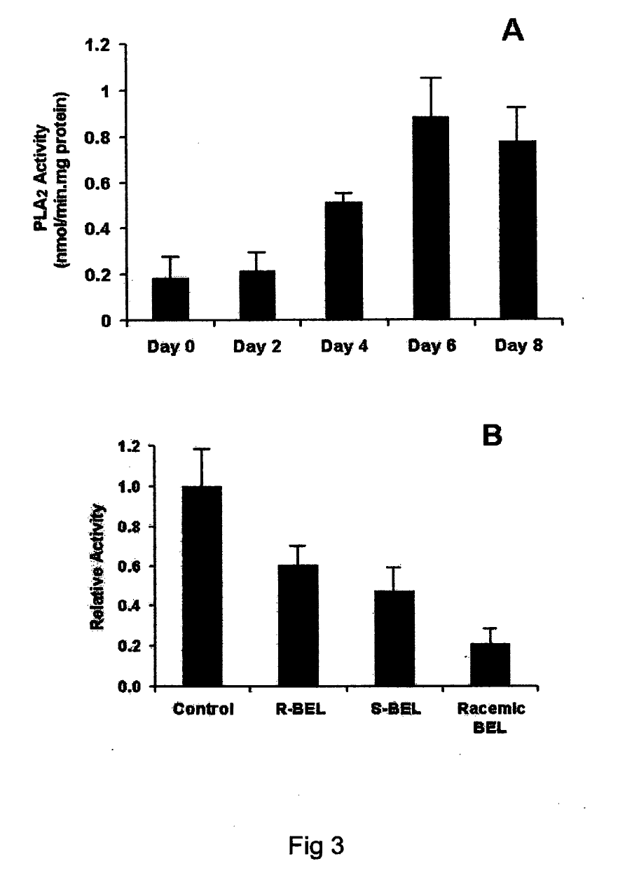

FIG. 3. Activities of PLA₂ in 3T3-L1 cells during differentiation and their inhibition by BELs. 3T3-L1 cells were cultured and induced to differentiation as described in “Test Procedures.” At indicated differentiation stages, cell homogenates were prepared as described in “Test Procedures.” Phospholipase A₂ activity was assessed by incubating 3T3-L1 cell protein (100-200 μg) with radiolabelled 1-palmitoyl-2-[1-¹⁴C]-oleoyl-sn-glycerol-3-phosphocholine (POPC) (50 mCi/mmol, 5 μM final concentration, introduced by ethanol injection (2 μL)) in assay buffer (final conditions: 100 mM Tris-HCl, 4 mM EGTA, pH=7.2) at 37° C. for 30 min in a final volume of 200 μL. Reactions were quenched by addition of butanol (100 μL). 30 microliters of the organic phase was spotted on a Whatman silica plate which was developed with a nonpolar acidic mobile phase (100 mL of 70/30/1 petroleum ether/ethyl ether/acetic acid). Spots corresponding to fatty acids were scrapped into scintillation vials and radioactivity was quantified by scintillation spectrometry as described previously. A, iPLA₂ activities of 3T3-L1 cells during differentiation. B, iPLA₂ activities of homogenates of day 8 3T3-L1 cells after incubation at room temperature for 3 minutes in the absence or presence of 10 μM of (R)-BEL, (S)-BEL or racemic BEL.

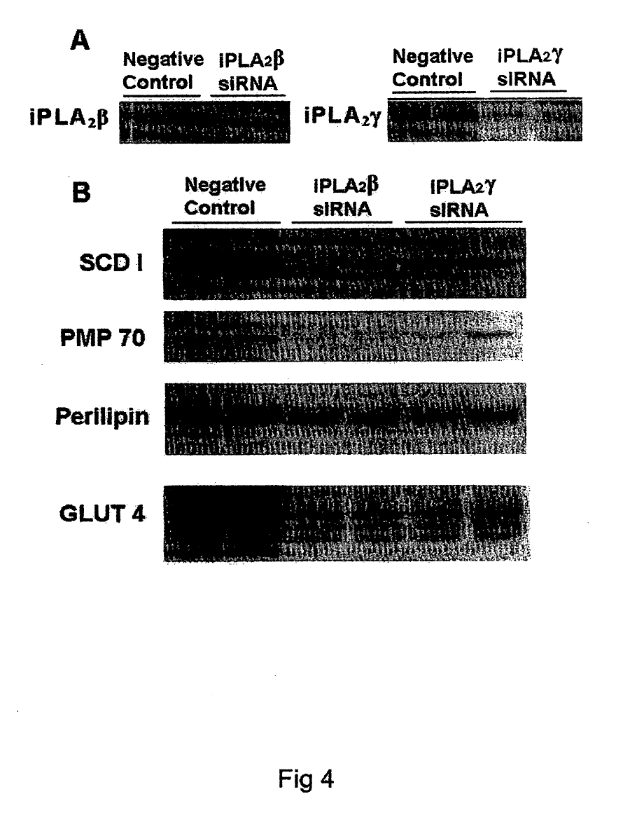

FIG. 4. Effects of siRNAs directed against iPLA₂β or iPLA₂γ on the expression of several adipocyte markers. 3T3-L1 cells were cultured and transfected with 20 nM negative control siRNA, siRNA directed against iPLA₂β or siRNA directed against iPLA₂γ prior to induction to differentiation as described in “Test Procedures.” Total protein extracts were prepared as described in “Test Procedures.” 40 μg proteins from 3T3-L1 cells were loaded to each lane, separated by SDS-PAGE and transferred to Immobilon-P membranes. Powdered milk (5% (w/v)) was used to block nonspecific binding sites prior to incubation with primary antibody directed against each specific protein as indicated. After incubation with horseradish peroxidase-conjugated secondary antibody, proteins were visualized by enhanced chemiluminescence as described in “Test Procedures.” A, Western blot analysis of day 4 3T3-L1 cell proteins using antibodies against iPLA₂β or iPLA₂γ. B, Western blot analysis of day 8 3T3-L1 cell proteins using antibodies against SCD I, PMP 70, perilipin, or GLUT4.

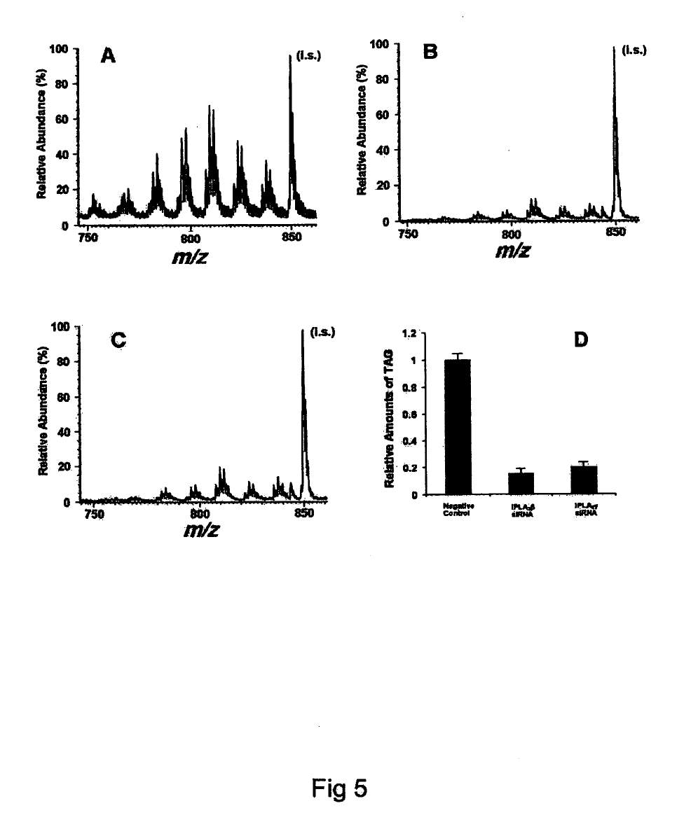

FIG. 5. Effects of siRNAs directed against iPLA₂β or iPLA₂γ on TAG accumulation during 3T3-L1 cell differentiation. 3T3-L1 cells were cultured and transfected with 20 nM negative control siRNA, siRNA directed against iPLA₂β, or siRNA directed against iPLA₂γ prior to induction to differentiation as described in “Test Procedures.” 3T3-L1 cells were grown to day 8 and the cell monolayer was washed with ice-cold PBS and scraped into 1 mL 50 mM LiCl. The lipids were extracted by the method of Bligh-Dyer⁽³³⁾ in the presence of an internal standard (Tril7:0TAG, 200 nmol/mg protein). Mass spectral analysis of TAG was performed by ESI/MS as described in “Test Procedures.” ESI/MS spectra of TAG of day 8 3T3-L1 cells with pretreatment of negative control siRNA (A), siRNA directed against iPLA₂β (B) or siRNA directed against iPLA₂γ (C) were shown. The results of TAG quantification (D) represent means ±S.E.M. of at least three independent cultures.

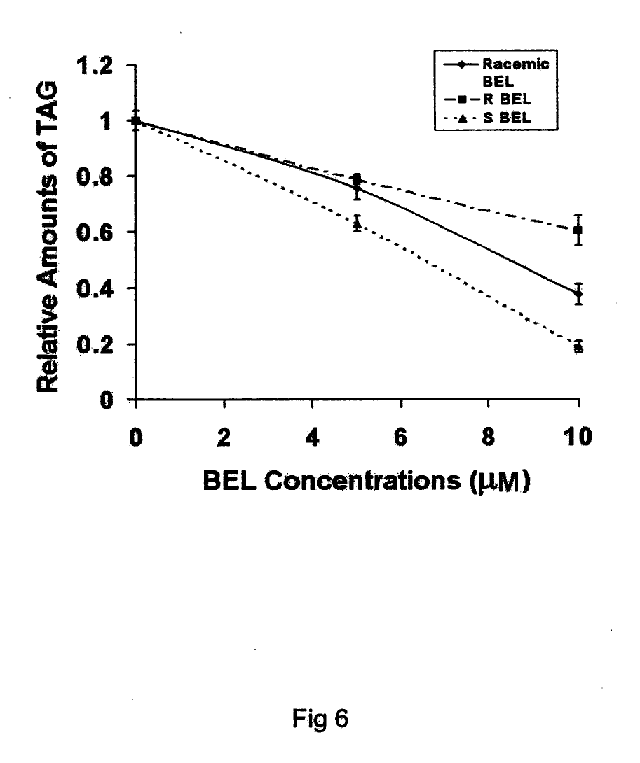

FIG. 6. Effects of BELs on TAG accumulation during hormone-induced differentiation of 3T3-L1 cells. 3T3-L1 cells on two days post confluent were washed three times with DMEM, incubated at 37° C. for 20 min in DMEM with 0, 5, 10 μM racemic, R-, or S-BEL. The cells were induced to differentiate as described in “Test Procedures” in the presence of indicated concentrations of BELs for 4 days. 3T3-L1 cells were grown to day 8 and the cell monolayer was washed with ice-cold PBS and scraped into 1 mL 50 mM LiCl. The lipids were extracted by the method of Bligh-Dyer⁽³³⁾ in the presence of an internal standard (Tril7:0TAG, 200 nmol/mg protein). Mass spectral analysis of TAG was performed by ESI/MS as described in “Test Procedures.” The results represent means ±S.E.M. of at least three independent cultures.

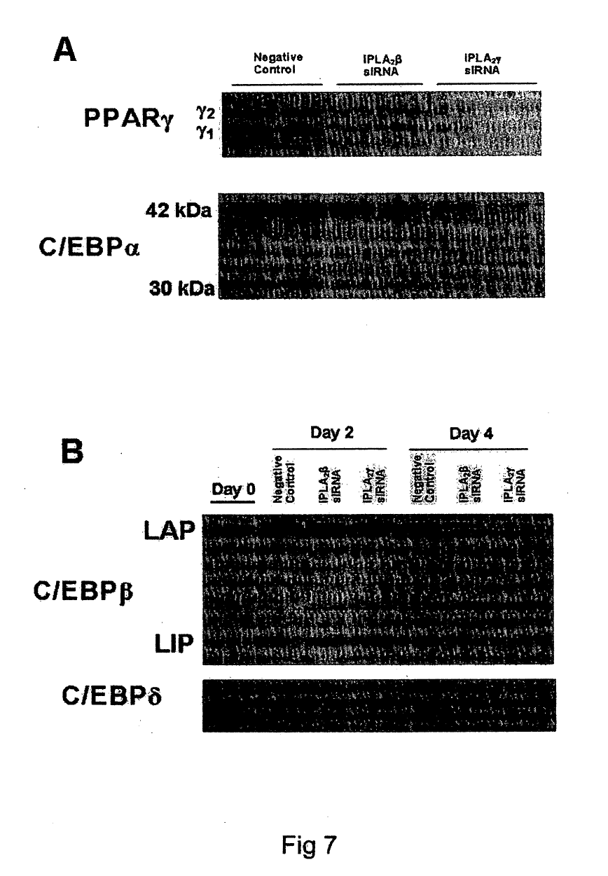

FIG. 7. Effects of siRNAs directed against iPLA₂β or iPLA₂γ on the expression of several transcription factors. 3T3-LI cells were cultured and transfected with 20 nM negative control siRNA, siRNA directed against iPLA₂β, or siRNA directed against iPLA₂γ prior to induction to differentiation as described in “Test Procedures.” At the indicated differentiation stages, nuclear extracts were prepared as described in “Test Procedures.” 20 μg of nuclear protein were loaded to each lane, separated by SDS-PAGE, and transferred to Immobilon-P membranes. Powdered milk (5% (w/v)) was used to block nonspecific binding sites prior to incubation with primary antibody directed against each specific protein as indicated. After incubation with horseradish peroxidase-conjugated secondary antibody, proteins were visualized by enhanced chemiluminescence as described in “Test Procedures.” A, Western blot analysis of day 8 3T3-L1 cell nuclear proteins using antibodies against C/EBPα and PPARγ. B, Western blot analysis of 3T3-L1 cell nuclear proteins using antibodies against C/EBPβ and C/EBPδ.

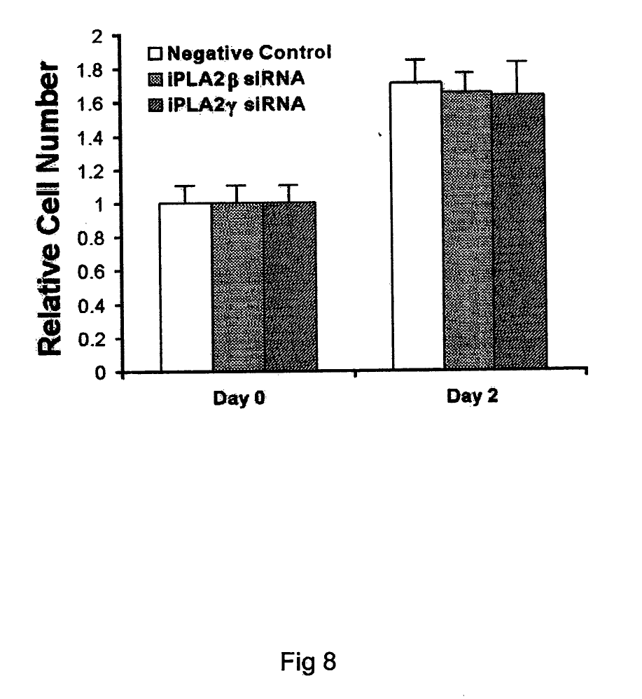

FIG. 8. Effects of siRNAs directed against iPLA₂β or iPLA₂γ on mitotic clonal expansion. 3T3-L1 cells were cultured and transfected with 20 nM negative control siRNA, siRNA directed against iPLA₂β, or siRNA directed against iPLA₂γ prior to induction to differentiation as described in “Test Procedures.” Cell numbers of day 2 3T3-L1 cells were counted and represented means ±S.E.M. of at least four independent cultures after normalization to the number of day 0 cells.

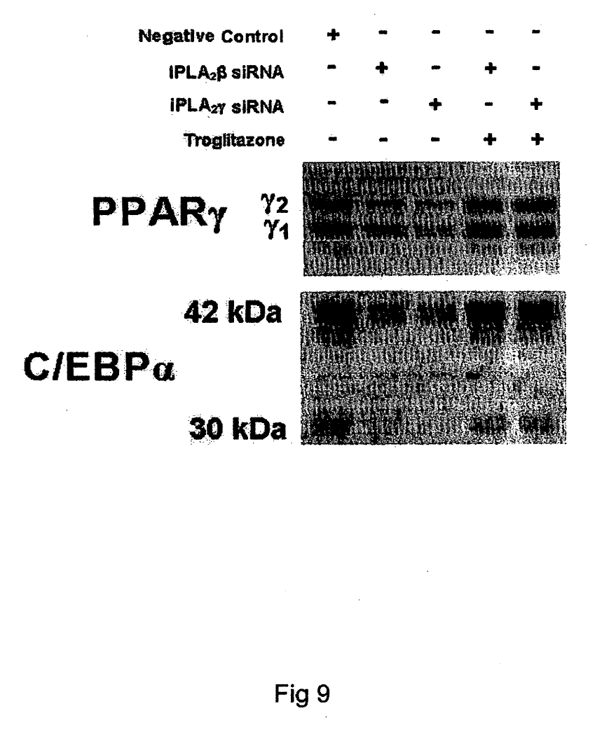

FIG. 9. Troglitazone rescues the expression of C/EBPα and PPARγ during the differentiation of 3T3-L1 cells pretreated with siRNAs directed against iPLA₂β or iPLA₂γ. 3T3-L1 cells were cultured and transfected with 20 nM negative control siRNA, siRNA directed against iPLA₂β, or siRNA directed against iPLA₂γ prior to induction to differentiation in the presence or absence of 10 μM troglitazone as described in “Test Procedures.” On day 8 of differentiation, nuclear extracts were prepared and 20 μg of protein were loaded to each lane, separated by SDS-PAGE and transferred to Immobilon-P membranes. Powdered milk (5% (w/v)) was used to block nonspecific binding sites prior to incubation with primary antibodies directed against C/EBPα or PPARγ. After incubation with secondary antibody, proteins were visualized by enhanced chemiluminescence as described in “Test Procedures.”

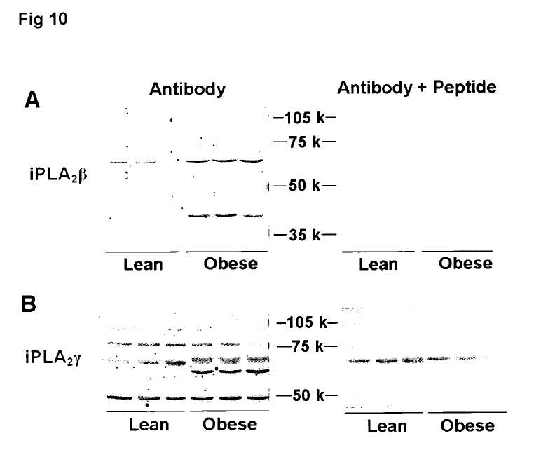

FIG. 10. Up-regulation of iPLA₂β and iPLA₂γ in obese Zucker (fa/fa) rat White Adipose Tissue. Proteins of WAT from obese Zucker (fa/fa) rats and their congenic lean controls were extracted as described in “Test Procedures.” 40 μg of protein were loaded to each lane, separated by SDS-PAGE and transferred to Immobilon-P membranes. Powdered milk (5% (w/v)) was used to block nonspecific binding sites prior to incubation with primary antibodies directed against iPLA₂β (A) and iPLA₂γ (B) in presence or absence of excess amounts (20 fold) of corresponding antigen peptides. After incubation with horseradish peroxidase-conjugated secondary antibody, proteins were visualized by enhanced chemiluminescence as described in “Test Procedures.”

### DETAILED DESCRIPTION OF THE INVENTION

This discovery relates to functional calcium-independent phospholipases A₂β and A₂γ and more particularly to useful effective research tools therefore and for methods of therapeutically intentionally controlling obesity in living animals by treating preadipocytes such as 3T3-L1 preadipocytes in the living animals or representative samples thereof. More particularly, the discovery relates to the discovery of and use of iPLA₂β and iPLA₂γ metabolic targets in a living animal and to inhibitors of the activity of those targets.

The inventor identifies for the first time iPLA₂β and iPLA₂γ as functional metabolic targets in a living system for beneficially modulating fat content of tissue associated therewith. The inventor also provides a method for modulating fat in a living system which comprises using a silencing gene(s) and/or pharmacological method(s) for modulating fat in a living system, comprising the administration of S-BEL, R-BEL or a racemic mixture thereof to a living system. Additional utility is present and in this discovery herein as a screening method useful for identifying drugs useful to modulate fat in a living system comprising at least one of a biologic method and a pharmacological method.

In this discovery, the inventor discovered dramatic up-regulation of both iPLA₂β or iPLA₂γ mRNA levels, protein content and enzymatic activities during hormone-induced differentiation of 3T3-L1 cells temporally coordinated with the down regulation of cPLA₂° C. to near-background levels. Moreover, the essential roles of iPLA₂β or iPLA₂γ in adipocyte differentiation and their interplay with C/EBP and PPAR transcription factors have been identified by the inventor specific siRNAs knockdown of either iPLA₂β or iPLA₂γ activity. My results demonstrate that functional down regulation of iPLA₂β or iPLA₂γ inhibits adipocyte differentiation via preventing PPARγ and C/EBPα expression without affecting the expression of C/EBPβ and C/EBPδ. Collectively, my results are the first to demonstrate the central roles of both iPLA₂β or iPLA₂γ in the differentiation of a mammalian preadipocyte cell line into adipocytes. My discovery provides a method for discovering and identifying agonists to such cellular differentiation including a method for determining and identifying a pharmacological inhibition and molecular biologic inhibition.

The inventor discovered a screening method and research tool for identifying discovery drugs which are useful to successfully hold weight in a living mammal or if desired to reduce weight gain or to reduce weight.

As used herein, the term “compound” includes cell(s), compounds, ions/anions, cations and salts.

As used herein, the term “adipocyte” includes any cell storing fat.

As used herein, the term “tissue” includes tissue, cells and collections of a multiplicity of homogenous or nearly homogenous cell lines or a sample thereof or a representative sample thereof. In an aspect the tissue is a living mammalian tissue such as in a tissue culture or living mammal or in a living transgenic mouse.

As used herein, the term “peptide” is any of a group of compounds comprising two or more amino acids linked by chemical bonding between their respective carboxyl and amino groups. The term “peptide” includes peptides and proteins that are of sufficient length and composition to affect a biological response, e.g. antibody production or cytokine activity whether or not the peptide is a hapten. The term “peptide” includes modified amino acids, such modifications including, but not limited to, phosphorylation, glycosylation, acylation, prenylation, lipidization and methylation.

As used herein, the term “polypeptide” is any of a group of natural or synthetic polymers made up of amino acids chemically linked together such as peptides linked together. The term “polypeptide” includes peptide, translated nucleic acid and fragments thereof.

As used herein, the term “polynucleotide” includes nucleotide sequences and partial sequences, DNA, cDNA, RNA variant isoforms, splice variants, allelic variants and fragments thereof.

As used herein, the terms “protein”, “polypeptide” and “peptide” are used interchangeably herein when referring to a translated nucleic acid (e.g. a gene product). The term “polypeptide” includes proteins.

As used herein, the term “isolated polypeptide” includes a polypeptide essentially and substantially free from contaminating cellular components.

As used herein, the term “isolated protein” includes a protein that is essentially free from contamination cellular components normally associated with the protein in nature.

As used herein, the term “nucleic acid” refers to oligonucleotides or polynucleotides such as deoxyribonucleic acid (DNA) and ribonucleic acid (RNA) as well as analogs of either RNA or DNA, for example made from nucleotide analogs any of which are in single or double stranded form.

As used herein, the term “patient” and subject” are synonymous and are used interchangeably herein.

As used herein, the term “expression” includes the functional and competent biosynthesis of a product as an expression product from a gene such as the transcription of a structural gene into mRNA and the translation of mRNA into at least one peptide or at least one polypeptide.

As used herein, the term “mammal” includes living animals including humans and non-human animals such as murine, porcine, canine and feline.

As used herein, the term “sample” means a viable sample of biological tissue or fluid. A biological sample includes representative sections of tissues of living animals or viable cells or cell culture.

As used herein, the term “antisense” means a strand of RNA whose sequence of bases is complementary to messenger RNA.

As used herein, the term “siRNA” means functional short interfering RNA. Articles which describe the effects of small interfering RNA (siRNA) on silencing genes are 1. Elbashir, S. M. et al (2001) Nature, 411, 494-498; 2. Hannon, G. J. (2002) Nature, 418, 244-251; and 3. Tijsterman, M. (2002) Annu. Rev. Genet., 36, 489-519. Instruction for siRNA construction is available from Silencer™siRNA Construction Kit Instruction Manual, Catalog #: 1620, Ambion Inc., 2130 Woodward St., Austin, Tex. 78744-1832, USA. Instruction for siRNA transfection is available in Silencer™siRNA Transfection Kit Instruction Manual, Catalog #: 1630, Ambion Inc., 2130 Woodward St., Austin, Tex. 78744-1832, USA. See http://www.ambion.com/techlib/tn/101/7.html.

The phrase “a sequence encoding a gene product” refers to a nucleic acid that contains sequence information, e.g., for a structural RNA such as rRNA, a tRNA, the primary amino acid sequence of a specific protein or peptide, a binding site for a transacting regulatory agent, an antisense RNA or a ribozyme. This phrase specifically encompasses degenerate codons (i.e., different codons which encode a single amino acid) of the native sequence or sequences which may be introduced to conform with codon preference in a specific host cell.

By “host cell” is meant a cell which contains an expression vector and supports the competent replication or expression of the expression vector. Host cells may be prokaryotic cells such as E. coli, or eukaryotic cells such as yeast, insect, amphibian, e.g. Xenopus, or mammalian cells such as HEK293, CHO, HeLa and the like.

As used herein, the term “administration” includes the effective administration which includes the application of a drug to a sample or to the body of a patient or research subject by injection, inhalation, ingestion, or any other effective means whereby the drug is presented to the target or area of intended delivery and reception of the drug. Normally after such administration the functional effects of the drug are detected as by suitable effective analytical means to determine the effect if any of the drug following its administration.

As used herein a “therapeutic amount” is an amount of a moiety such as a drug or compound which produces a desired or detectable therapeutic effect on or in a mammal administered with the moiety.

The term “recombinant” when used with reference to a cell, or protein, nucleic acid, or vector, includes reference to a cell, protein, or nucleic acid, or vector, that has been modified by the introduction of a heterologous nucleic acid, the alteration of a native nucleic acid to a form not native to that cell, or that the cell is derived from a cell so modified. Thus, for example, recombinant cells express genes and proteins that are not found within the native (non-recombinant) forms of the cell or express native genes that are otherwise abnormally expressed, under expressed or not expressed at all.

As used herein, an “expression vector” means a nucleic acid construct, generated recombinantly or synthetically, with a series of specific nucleic acid elements which permit competent transcription of a particular nucleic acid in a host cell. The expression vector can be part of a plasmid, virus, or nucleic acid fragment. Typically, the expression vector includes a nucleic acid to be transcribed operably linked to a promoter.

The phrase “functional effect” in the context of assays for testing compounds affecting fat content includes the determination of any parameter that is indirectly or directly under the influence of the administration of the silencing gene or a compound.

By “determining the functional effect” or effect is meant examining the effect of a compound or negative control siRNA that increases or decreases pre-adipocyte differentiation.

Human iPLA₂β: See Larsson Forsell P. K., Kennedy, B. P., Claesson H. E. (1999) Eur J. Biochem. 262, 575-85. This reference is incorporated herein in its entirety by reference.
iPLA₂γ: See Mancuso, D. J., Jenkins, C. M., and Gross, R. W. (2000) J Biol Chem 275, 9937-9945 and Mancuso, D. J., Jenkins, C. M., Sims, H. F., Cohen, J. M., Yang, J., and Gross, R. W. (2004) Eur J Biochem 271, 4709-4724. Both of these immediately aforelisted two references are incorporated herein in their entirety by reference.

Mouse iPLA₂β: See NCBI GenBank (public available sequence database) Accession Numbers (Nucleotide and Amino Acid Translation): BC049778 and BC057209.

Mouse iPLA₂γ: See NCBI GenBank (public available sequence database) Accession Number (Nucleotide and Amino Acid Translation): NM_{—}026164.

Alterations in lipid secondary messenger generation and lipid metabolic flux are essential in promoting the differentiation of adipocytes. To determine the specific types and subtypes of intracellular phospholipases facilitating hormone-induced differentiation of 3T3-L1 cells into adipocytes, I examined alterations in the mRNA level, protein mass, and activity of the previously characterized mammalian intracellular phospholipases A₂. Hormone-induced differentiation of 3T3-L1 cells resulted in a 7.3±0.5 and 7.4±1.4 fold increase of mRNA encoding the calcium independent phospholipases, iPLA₂β and iPLA₂γ, respectively. In contrast, the temporally coordinated loss of at least 90% of mRNA encoding cPLA₂α was manifest. Western analysis demonstrated the near absence of both iPLA₂β and iPLA₂γ protein mass in resting 3T3-L1 cells which increased dramatically during differentiation. In vitro measurement of calcium-dependent and calcium-independent phospholipase activities demonstrated an increase in both iPLA₂β and iPLA₂γ activities which were discriminated using the chiral mechanism based inhibitors (S)- and (R)-BEL, respectively. Remarkably, treatment of 3T3-L1 cells with siRNA directed to either iPLA₂β or iPLA₂γ resulted in the failure of 3T3-L1 cells to undergo hormone-induced differentiation. Moreover, analysis of the temporally programmed expression of transcription factors demonstrated that the siRNA knockdown of iPLA₂β or iPLA₂γ resulted in the failure of 3T3-L1 cell differentiation from the down regulation of the expression of PPARγ and the CCAAT enhancer binding protein α (C/EBPα). No alterations in the expression of the early stage transcription factors C/EBPβ and C/EBPδ were observed. Collectively, these results demonstrate prominent alterations in the type and magnitude of intracellular phospholipases A₂ during 3T3-L1 cell differentiation into adipocytes and identify the requirement of iPLA₂β and iPLA₂γ for the adipogenic program which drives resting 3T3-L1 cells into adipocytes after hormone stimulation.

Recent analyses of the transcriptional programs utilized for adipocyte differentiation have identified the critical roles of the CCAAT/enhancer-binding protein (C/EBP) family and the peroxisome proliferator activated receptor (PPAR) γ in mediating the transcriptional alterations required for adipocyte differentiation^{(3,10)}. Hormone induced growth-arrested 3T3-L1 cells treated with by insulin, MIX and dexamethasone express the early transcription factors C/EBPβ and C/EBPδ which lead to their reentry into the cell cycle^{(25,26)}. C/EBPβ and C/EBPδ then activate the transcription of C/EBPα and PPARγ, which are believed to be both antimitotic and to act synergistically to activate the expression of adipocyte specific genes leading to the differentiated adipocyte phenotype^{(27,28).}

In this discovery, the inventor demonstrated the dramatic up-regulation of both iPLA₂β and iPLA₂γ mRNA levels, protein content and enzymatic activities during hormone-induced differentiation of 3T3-L1 cells temporally coordinated with the down regulation of cPLA₂α to near-background levels. Moreover, the essential roles of iPLA₂β and iPLA₂γ in adipocyte differentiation and their interplay with C/EBP and PPAR transcription factors have been identified by specific siRNAs knockdown of either iPLA₂β or iPLA₂γ activity. The results demonstrate that down regulation of iPLA₂β or iPLA₂γ inhibits adipocyte differentiation via preventing PPARγ and C/EBPα expression without affecting the expression of C/EBPβ and C/EBPδ. Collectively, these results are the first to demonstrate the central roles of both iPLA₂β and iPLA₂γ in the differentiation of a mammalian preadipocyte cell line into adipocytes.

In an aspect, the specific mammalian dose of an inhibitor or chemical is an effective amount according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. The amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Formulations employed herein will generally be aqueous based, will include compositions of pharmaceutical grade and purity.

Compounds used will likely be in a pharmacologically effective amount pharmaceutical grade preferred, (including immunologically reactive fragments) are administered to a subject such as to a living patient using standard effective administration techniques, preferably peripherally (i.e. not by administration into the central nervous system) by intravenous, intraperitoneal, subcutaneous, pulmonary, transdermal, intramuscular, intranasal, buccal, sublingual, or suppository administration.

The compositions for effective administration are designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners.

The following examples are illustrative are not meant to be limiting of the discovery in any way.

Test Procedures

Materials:

3T3-L1 cells were obtained from ATCC (Manassas, Va.). Fetal calf serum and DMEM were purchased from Life Technologies, Inc. (Rockville, MD). Fetal bovine serum was obtained from BioWhittaker, Inc. (Walkersville, Md.). Reagents for reverse transcription and quantitative polymerase chain reaction (PCR) were supplied from Applied Biosystem (Foster City, Calif.). siRNA construction and transfection kits were purchased from Ambion (Austin, TA). All radiolabeled lipids were obtained from American Radiolabeled Chemicals Inc. (St. Louis, Mo.). Most other chemicals were obtained from Sigma Chemical Co. (St. Louis, Mo.). Anti-PPARγ, anti-C/EBPα, anti-C/EBPβ, anti-C/EBPδ, anti-SCD I and anti-cPLA₂α antibodies were obtained from Santa Cruz Biotechnology, Inc. (Santa Cruz, Calif.). Anti-perilipin and anti-GLUT4 antibodies were kindly provided by Dr. Perry E. Bickel (Washington University, St. Louis). Anti-PMP70 antibody was obtained from Affinity Bioreagent (Golden, Colo.). Anti-iPLA₂β or anti-iPLA₂γ polyclonal antibodies were produced utilizing the synthetic peptides CEFLKREFGEHTKMTDVKKP (iPLA₂β) or CENIPLDESRNEKLDQ (iPLA₂γ) and immuno-affinity purified as previously described (29).

Cell Culture of 3T3-L1 Cells and Differentiation into the Adipoctye Phenotype

3T3-L1 cells 1 were cultured to confluence in Dulbecco's modified Eagle's medium (DMEM) containing 10% calf serum (CS) by changing the medium every two days as previously described⁽³⁰⁾. Two days after cell confluence, differentiation was initiated by adding differentiation medium 1 (0.5 mM MIX, 0.25 μM dexamethasone, 1 μg/mL insulin in DMEM containing 10% fetal bovine serum (FBS)). Two days later, MIX and dexamethasone were removed and insulin (1 μg/mL) was maintained for two more days. Thereafter, cells were grown in DMEM containing 10% FBS in the absence of differentiating reagents by replacing the media every two days.

Reverse Transcription and Quantitative Polymerase Chain Reaction (PCR)

Total RNA was purified from 3T3-L1 cell pellets utilizing a RNeasy® Mini Kit from Qiagen (28159 Avenue Stanford, Valencia, Calif., 9155 USA) according to the manufacturer's instructions (Catalog #74104). For cDNA preparation, 250 pmol of random hexamers were hybridized by incubation for 10 min at 25° C. and extended by incubation for 30 min at 48° C. in the presence of 125 units of reverse transcriptase in 100 μL of PCR buffer (5.5 mM MgCl₂, 0.5 mM of each dNTP, and 40 units of Rnase inhibitor). Reverse transcriptase was inactivated by incubation at 95° C. for 5 min. Amplification of each target cDNA was performed with TaqMan® PCR reagent kits and quantified by the ABI PRISM 7700 detection system according to the protocol provided by the manufacturer (Applied Biosystems, Foster City, Calif.). A traditionally utilized standard gene, GAPDH, was measured and used as internal standard.

Oligonucleotide primer pairs and probes specific for cPLA₂α (5′-CCTTTGAGTTCATTTTGGATCCTAA/5′-TGTAGCTGTGCCTAGGGTTTCA T/5′-AGGAAAATGTTTTGGAGATCACACTGATGGATG), iPLA₂β (5′-CCTTCCATTACGCTGTGCAA/5′-GAGTCAGCCCTTGGTTGTT/5′-CCAGGTGCTACAGCTCCTAGGAAAGAATGC) and iPLA₂γ (5′-GAGGAGAAA AAGCGTGTGCTACTTC/5′ GGTTGTTCTTCTTAAGGCCTGAA/5′ TCTGTTATCAATACTCACTCTTGCAATA) were employed.

Protein Extraction and Western Blot

Protein from 3T3-L1 cells were extracted as described previously⁽³¹⁾. Briefly, the cell monolayer was washed with ice cold PBS and subsequently scraped into 1 mL ice cold lysis buffer (50 mM Tris.HCl, PH7.4, 150 mM NaCl, 1 mM EDTA, 0.25% sodium deoxycholate, 1% Nonidet P-40, 0.1% SDS, 1 mM phenylmethylmethanesulfonyl fluoride, 2 μg/mL aprotinin and 1 μg/mL leupeptin). The solution was incubated on ice for 10 min after vortexing for 10 s. The cell homogenate was spun at 10,000×g at 40° C. in a tabletop centrifuge for 10 min and the supernatant was transferred to a tube carefully and stored at −70° C. until used for Western blot analysis. Nuclear extracts were prepared with NE-PER® Nuclear and Cytoplasmic Extraction Reagents from Pierce (Rockford, Ill., USA) according to manufacturer's protocol. Proteins were separated by SDS-PAGE and transferred to Immobilon-P membranes (Millipore, Billerica, Mass., USA) in 10 mM CAPS buffer (pH=11) containing 10% methanol. Powdered milk (5% (w/v)) was used to block nonspecific binding sites prior to incubation with primary antibody directed against each specific protein as indicated. After incubation with secondary antibody (IgG-HRP conjugate diluted 1:5000 in blocking buffer), proteins were visualized by enhanced chemiluminscence according to the instructions of the manufacturer (Amersham Bioscience, Piscataway, N.J.).

On the day of the test, the media of 3T3-L1 cells at different stages of differentiation were removed. The cells were washed briefly with PBS and detached by incubation in trypsin-EDTA (0.25% w/v) at 37° C. for 5 mm. The cells were washed again with 5 volumes of CMRL-1066, transferred to a 50 mL Falcon centrifuge tube and centrifuged for 5 min at 1700 rpm at 4° C. The resulting cell pellets were resuspended in CMRL-1066 medium and centrifuged as above three times. The cell pellets from 4 plates (10 mm diameter) were resuspended in 3 ml lysis buffer (0.25 M sucrose, 25 mM imidazole, pH=7.2) and were sonicated six times for 1 s each. The tubes were placed on ice for 3 min followed by repeated sonication. Phospholipase A₂ assays were performed as described previously⁽³²⁾. Briefly, phospholipase A₂ activity was assessed by incubating 3T3-L1 cell protein (100-200 μg) with radiolabelled phosphatidylcholine, L-α-palmitoyl-2-oleoyl [oleoyl-1-¹⁴C] (POPC) (50 mCi/mmol, 5 μM final concentration, introduced by ethanol injection (2 μL)) in assay buffer (final conditions: 100 mM Tris-HCl, 4 mM EGTA, pH=7.2) at 37° C. for 30 mm in a final volume of 200 μL. Reactions were quenched by addition of butanol (100 μL). 30 microliters of the organic phase was spotted on a Whatman silica plate which was developed with a nonpolar acidic mobile phase (100 mL of 70/30/1 petroleum ether/ethyl ether/acetic acid). Spots corresponding to fatty acids were scrapped into scintillation vials and radioactivity was quantified by scintillation spectrometry as described previously⁽³²⁾. BEL enantiomers were resolved by chiral HPLC as described previously⁽³²⁾. In the inhibition assays of iPLA₂ by BEL, proteins were incubated with 10 μM (R)-BEL, (S)-BEL, racemic BEL or ethanol vehicle for 3 min at 22° C. prior to the addition of radiolabelled substrate.

siRNA Construction and Transfection

The siRNAs directed against iPLA₂β and iPLA₂γ were constructed employing the Silencer™ siRNA construction kit (Ambion, Austin, Tex., USA) according to the protocol provided by manufacturer. Upon confluence, the 3T3-L1 cell media were changed to growth media without antibiotics. One to two days later, cells were transfected with siRNAs (20 nM) using the siPORT™ lipid transfection reagent (Ambion) according to Ambion's instructions (See Catalogs 1620 and 1630, Ambion, Inc.). Five volumes of 1.2× differentiation medium 1 without antibiotics were added 4 hours after transfection and the cells were maintained at normal growing conditions and induced to differentiate as described above. Among four siRNAs for each targeting gene, the sequences specific for iPLA₂β (5′-AACAGCACAGAGAAUGAGGAG-3′) and iPLA₂γ (5′-AAGAUAAACAGCUUCAGGACA-3′) were selected based upon their potency to inhibit target gene expression. A scrambled siRNA was used as a negative control.

Triglyceride Extraction and Electrospray Ionization Mass Spectrometry

After siRNA transfection, 3T3-L1 cells were grown to day 8 as described above. The cell monolayer was washed with ice-cold PBS and scraped into 1 mL 50 mM LiCl. The lipids were extracted by the method of Bligh-Dyer⁽³³⁾ in the presence of an internal standard (Tril7:0TAG, 200 nmol/mg protein). Mass spectral analysis of TAG was performed by electrospray ionization utilizing a Finnigan TSQ Quantum spectrometer (Finnigan MAT, San Jose, Calif.) as previously described⁽³⁴⁾.

Protein Extraction from White Adipocyte Tissue of Zucker Rats

Female obese Zucker (fa/fa) rats and lean congenic controls (5-6 weeks old) were housed and maintained with a 12-hr light/12-hr dark photoperiod. Water and foods were given ad libitum. Animals were killed and inguinal fat pads (white adipose tissue) were removed, rapidly frozen in liquid nitrogen and grinded with a motor and pestle. To the tissue powder was added lysis buffer (50 mM Tris-HCl, pH=7.4, 150 mM NaCl, 1 mM EDTA, 0.25% sodium deoxycholate, 1% Nonidet P-40, 0.1% SDS, 1 mM phenylmethylmethanesulfonyl fluoride, 2 μg/mL aprotinin and 1 μg/mL leupeptin) and the resulting mixtures were homogenized with a Potter-Elvehjem apparatus. The homogenates were spun at 10,000×g at 4° C. in a tabletop centrifuge for 10 min and the supernatant was transferred to a tube carefully and stored at −70° C. until used for Western blot analysis.

Protein concentration was determined with a BCA protein assay kit (Pierce, Rockford, Ill.) using bovine serum albumin (BSA) as a standard. All data were normalized to protein content and are presented as the mean ±SEM. Statistically significant differences between mean values were determined using an unpaired Student's t tests.

Results

Alterations in lipid secondary messenger generation and lipid metabolic flux are essential in promoting the differentiation of adipocytes. To determine the specific types and subtypes of intracellular phospholipases facilitating hormone-induced differentiation of 3T3-L1 cells into adipocytes, I examined alterations in the mRNA level, protein mass, and activity of the previously characterized mammalian intracellular phospholipases A₂. Hormone-induced differentiation of 3T3-LI cells resulted in a 7.3±0.5 and 7.4±1.4 fold increase of mRNA encoding the calcium independent phospholipases, iPLA₂β and iPLA₂γ, respectively. In contrast, the temporally coordinated loss of at least 90% of mRNA encoding cPLA₂α was manifest. Western analysis demonstrated the near absence of both iPLA₂β and iPLA₂γ protein mass in resting 3T3-L1 cells which increased dramatically during differentiation.

In vitro measurement of calcium-dependent and calcium-independent phospholipase activities demonstrated an increase in both iPLA₂β and iPLA₂γ activities which were discriminated using the chiral mechanism based inhibitors (S)— and (R)-BEL, respectively. Measurable calcium-dependent activity decreased dramatically in day 8 cells. Remarkably, treatment of 3T3-L1 cells with siRNA directed to either iPLA₂β or iPLA₂γ resulted in the failure of 3T3-L1 cells to undergo hormone-induced differentiation. Moreover, analysis of the temporally programmed expression of transcription factors demonstrated that the siRNA knockdown of iPLA₂β or iPLA₂γ resulted in the failure of 3T3-L1 cell differentiation from the down regulation of the expression of PPARγ and the CCAAT enhancer binding protein α (C/EBPα). No alterations in the expression of the early stage transcription factors C/EBPβ and C/EBPδ were observed.

Collectively, these results demonstrate prominent alterations in the type and magnitude of intracellular phospholipases A₂ during 3T3-L1 cell differentiation into adipocytes and identify the requirement of iPLA₂β and iPLA₂γ for the adipogenic program which drive resting 3T3-L1 cells into adipocytes after hormone stimulation.

Results

Alterations in the mRNA Levels of Intracellular Phopspholipases A₂ During Differentiation of 3T3-L1 Preadipocytes

Research work has underscored the essential roles of eicosanoid metabolites and LPC derived LPA in adipocyte differentiation⁽¹⁹⁻²²⁾. Since these metabolites are all downstream products of phospholipase A₂ catalyzed reactions, I sought to determine the specific types and amounts of phospholipase A₂ mRNA, protein and activity corresponding to each of the previously characterized mammalian intracellular phospholipases A₂ as a function of time after hormone-induced differentiation of 3T3-L1 preadipocytes. In resting cells, cPLA₂α mRNA was prominent, with only minimal amounts of mRNA encoding calcium independent phospholipases A₂ detectable (FIG. 1). However, after hormone-induced differentiation, the levels of cPLA₂α mRNA decreased dramatically to near background levels (FIG. 1A). Remarkably, the levels of iPLA₂β and iPLA₂γ mRNA increased 7.3±0.5 and 7.4±1.4 fold, respectively (FIGS. 1B and 1C). Collectively, these results demonstrate the dramatic and temporally coordinated changes in the mRNA levels of each of the previously characterized mammalian intracellular phospholipases A₂ during adipocyte differentiation.

Alterations of Intracellular Phospholipase A₂ Protein Mass and Activity during Differentiation of 3T3-L1 Preadipocytes

To further substantiate the functional importance of the observed alterations in mRNA levels, western blot analysis was performed. Western analyses demonstrated a decrease in cPLA₂α protein mass to near background levels (as predicted by the decreased contant of cPLA₂α mRNA in the differentiating adipocyte) and the dramatic increases of both iPLA₂β and iPLA₂γ (as predicted by increased mRNA levels encoding iPLA₂β and iPLA₂γ from quantitative PCR) (FIG. 2). The temporal course of the increased amounts of iPLA₂β and iPLA₂γ protein and the decreased amount of cPLA₂α protein were coordinately regulated (FIG. 2). Thus the protein mass of each intracellular phospholipase A₂ closely paralleled the intrinsic mRNA levels of each of the three mammalian intracellular phospholipases previously characterized in overexpressing systems (i.e. cPLA2₂α, iPLA₂β and iPLA₂γ). Collectively, these results demonstrate the importance of transcriptional regulation in modulating reciprocal alterations in specific classes of intracellular phospholipases A₂ during adipocyte differentiation.

To further investigate if alterations in the protein content of iPLA₂β and iPLA₂γ present during differentiation of 3T3-L1 cells were paralleled by changes in their activities, phospholipase A₂ assays were performed. During adipocyte differentiation iPLA₂ activity increased ≈4 fold (FIG. 3A). As anticipated, the measured increase in iPLA₂ activity was inhibited by mechanism-based inhibitor, racemic BEL (FIG. 3B). Previously, I demonstrated that (S)-BEL was approximately one order of magnitude more selective for iPLA₂β in comparison to iPLA₂γ, while (R)-BEL was approximately an order of magnitude more selective for iPLA₂γ⁽³²⁾. The measured iPLA₂ activity in 3T3-L1 adipocyte homogenate was inhibited to similar levels by either (S)-BEL or (R)-BEL (FIG. 3B) demonstrating that both iPLA₂β and iPLA₂γ contribute similarly to the total amounts of measured iPLA₂ activity in differentiated adipocytes.

Pretreatment of siRNAs Targeting iPLA₂β and iPLA₂γ Inhibits Hormone-induced Differentiation of 3T3-L1 Preadipocytes

These results, in the context of prior work on the importance of eicosanoids and lysolipids in adipocyte differentiation, suggested that calcium-independent PLA₂ activities may be required to promote adipocyte differentiation. To determine if iPLA₂β and iPLA₂γ are required for adipocyte differentiation, confluent 3T3-L1 cells were transfected with siRNA targeting iPLA₂β and iPLA₂γ. The efficiency of siRNA knockdown was judged by the iPLA₂β and iPLA₂γ protein levels at day 4 when iPLA₂s typically begin to accumulate (FIG. 4A). On day 8 of differentiation, cell pellets were scraped and the lipids were extracted for ESI/MS analysis. Treatment with siRNA directed against iPLA₂β and iPLA₂γ largely prevented the expression of iPLA₂β and iPLA₂γ proteins. In contrast, treatment with scrambled siRNA was without effect. Quantification of TAG using ESI/MS demonstrated that the accumulation of TAG during 3T3-L1 cells was greatly diminished after knockdown of iPLA₂β and iPLA₂γ (FIG. 5). Next, I examined the effect of iPLA₂β and iPLA₂γ siRNAs on several adipocyte specific protein markers by immunoblot analysis. Western analysis demonstrated the depression of SCD-I, perilipin, GLUT 4 and PMP 70 after knockdown of iPLA₂β and iPLA₂γ (FIG. 4B). These results indicated the requirement of iPLA₂β and iPLA₂γ for generation of the adipocyte phenotype. To substantiate the importance of iPLA₂β and iPLA₂γ in adipocyte differentiation utilizing an independent approach, chiral mechanism-based inhibition was employed. Treatment of 3T3-L1 cells with either (R) or (S)-BEL substantially decreased adipocyte differentiation (FIG. 6). Collectively these results demonstrate the importance of iPLA₂β and iPLA₂γ in the adipocyte differentiation process by independent genetic and pharmacological approaches.

PPARγ and C/EBPα are believed to be prominent effectors of the genetic programs which induce the expression of adipocyte specific genes leading to the development of the mature adipocytes^{(9,13,35,36)}. To explore the mechanism of inhibition of adipocyte differentiation imposed by knockdown of iPLA₂β and iPLA₂γ, I next examined the effect of siRNA directed against iPLA₂β or iPLA₂γ on the expression of PPARγ and C/EBPα. Nuclear extracts from day 8 hormone-induced 3T3-L1 cells after pretreatment with negative control siRNA, siRNA directed against iPLA₂β, or siRNA directed against iPLA₂γ were analyzed for alterations in the expression of PPARγ and C/EBPα by immunoblot analysis. The expression of both PPARγ and C/EBPα were greatly down-regulated after transfection with iPLA₂β or iPLA₂γ siRNAs (FIG. 7A). Thus, knockdown of iPLA₂β and iPLA₂γ inhibited adipocyte differentiation by preventing the expression of the proadipogenic transacting factors PPARγ and C/EBPα.

Next, the roles of iPLA₂β and iPLA₂γ in the hormone-induced differentiation of 3T3-L1 cells were characterized by examination of the initial induction of the early transcription factors C/EBβ, and C/EBPδ. Both C/EBβ, and C/EBPδ are essential in eliciting the expression of PPARγ, which in turn leads to the induction of the expression of C/EBPα^{(10,37,38)}. To investigate if the down regulation of PPARγ and C/EBPα by silencing iPLA₂β or iPLA₂γ was mediated by C/EBPβ and C/EBPδ, nuclear extracts from early stage hormone-induced 3T3-L1 cells pretreated with negative control siRNA, or siRNA directed against either iPLA₂β and iPLA₂γ were prepared. Imunoblot analysis demonstrated that the induced expression of C/EBPβ and C/EBPδ was not attenuated by pretreatment with siRNA directed against iPLA₂β and iPLA₂γ (in contrast to PPARγ and C/EBPα) (FIG. 7B). Moreover, the expression of liver-enriched inhibitory protein (LIP) isoform of C/EBPβ, which arises from utilization of an alternative translation initiation site and is believed to be a dominant-negative regulator of C/EBP family members⁽³⁹⁾, was also not affected by siRNAs directed toward iPLA₂β or iPLA₂γ (FIG. 7B). Previous work has demonstrated the requirement of C/EBPβ for mitotic clonal expansion during adipogenesis^{(25,26)}. The present results demonstrate that hormone-induced early stage mitotic clonal expansion was not affected by pretreatment with siRNA directed against iPLA₂β or iPLA₂γ (FIG. 8). Collectively, these results suggest that the down-regulation of iPLA₂β or iPLA₂γ does not prevent PPARγ and C/EBPα expression by affecting the expression of C/EBPβ and C/EBPδ but that these enzymes are essential for the activation of pathways at or proximal to the expression of PPARγ and C/EBPα.

Troglitazone Rescues Adipocyte Differentiation in iPLA₂β or iPLA₂γ siRNA Pretreated 3T3-L1 Cells

To further determine whether the inhibitory effects of iPLA₂β or iPLA₂γ siRNA on adipocyte differentiation were specifically caused by prevention of the expression and down stream effectors of PPARγ and C/EBPα, or alternatively if iPLA₂β or iPLA₂γ knockdown precluded cellular differentiation by other agonists, pharmacologic activation of PPARγ by troglitazone in the presence of iPLA₂ knockdowns were examined. Cultures of 3T3-L1 cells were pretreated with either siRNA against iPLA₂β or siRNA against iPLA₂γ, and incubated in differentiation media in the presence or absence of 10 μM troglitazone. On day 8 of differentiation, nuclear extracts were prepared and proteins were analyzed by immunobloting. Troglitazone rescued the expression of PPARγ and C/EBPα (FIG. 9) in the presence of siRNA directed against either iPLA₂β or iPLA₂γ and allowed completion of differentiation process after treatment of siRNA directed against iPLA₂β or iPLA₂γ. These results support the notion that knockdown of iPLA₂β or iPLA₂γ inhibited adipocyte differentiation by preventing the transcription programs mediated by PPARγ activation and was not the result of preventing the cell's ability to differentiate under appropriate activating conditions. Collectively, these results demonstrate that treatment of preadipocytes with siRNA directed against iPLA₂β or iPLA₂γ can be rescued by provision of a synthetic ligand of PPARy. Since PPARγ is activated by LPA derived from LPC⁽²⁴⁾, as well as fatty acids which may be released by iPLA₂β or iPLA₂γ, these results strongly suggest that both iPLA₂β and iPLA₂γ can provide the necessary lipid precursors for PPARγ activation.

Temporarily Coordinated Generation of the Alterations of iPLA₂β and iPLA₂γ Expression Levels in the Zucker Obese Rat

Dysregulation of a gene in the obese state provides important clues on the functional relevance of the gene in obese state and the mechanism contributing to obesity in that model. Up-regulation of iPLA₂β and iPLA₂γ and requirement of these two phospholipase proteins for adipogenesis in hormone-induced differentiation of 3T3-L1 cells suggest that they may be involved in the development of the disease state in obesity. Accordingly, I investigated the modulation of iPLA₂β and iPLA₂γ expression levels in Zucker (fa/fa) obese rats. 5-week-old female lean and homozygous obese rats were fed ad libitum. Animals were killed and inguinal fat pads (white adipose tissue) were removed for protein extraction. Protein extracts were analyzed for alterations in the expression of iPLA₂β and iPLA₂γ by immunoblot analysis. Western blot of iPLA₂β showed the dramatic up-regulation of the 65 kDa and 40 kDa protein product(s) in obese animals relative to their congenic lean controls in white adipocyte tissue. The identity of the 65 kDa band was substantiated by blocking immunoreactivity by preincubating the antibody solution with excess amounts of antigen peptide (FIG. 10A). Similarly, the expression level of 63 kDa iPLA₂γ was also dramatically induced in the WAT of Zucker obese rats in comparison to that of lean control (FIG. 10B). Interestingly, the level of 48 kDa iPLA₂γ proteolytic product was not altered. The identities of both 63 kDa and 48 kDa bands were further substantiated by blocking the antibody in the presence of excess amounts of peptide antigen (FIG. 10B). Collectively, these results demonstrate the dramatic changes in iPLA₂β and iPLA₂γ regulation in a standard genetic model of obesity.

Discussion:

My present discovery provides multiple independent lines of evidence that iPLA₂β and iPLA₂γ are essential regulatory components in the hormone-induced transcriptional programs which mediate the differentiation of 3T3-L1 cells into adipocytes.

I demonstrated the up-regulation of iPLA₂β and iPLA₂γ mRNA, protein mass and enzymatic activity after hormone-induced differentiation of 3T3-L1 preadipocytes.

Pharmacological inhibition of iPLA₂β or iPLA₂γ by chiral mechanism-based inhibition resulted in the inhibition of adipocyte differentiation as assessed by the suppression of the appearance of multiple markers of mature adipocytes.

Knockdown of iPLA₂ or iPLA₂γ by siRNA resulted in the ablation of hormone-induced differentiation of 3T3-L1 cells as assessed by multiple independent markers of adipocyte transcriptional programs and alterations in cellular lipid content.

Even in the presence of pharmacological inhibition by BEL or molecular biologic inhibition by siRNA knockdown, the cells could differentiate in the presence of troglitazone demonstrating that the functional integrity of processes downstream of PPARγ activation was not fundamentally comprised.

Collectively, my results strongly support the essential role of the iPLA₂ family of enzymes in facilitating the maturation of 3T3-L1 cells into adipocytes. Since prior studies have demonstrated the importance of eicosanoid metabolites^{(19,21)}, lysolipids^{(22,24)} and altered adipocyte calcium ion homeostasis^{(20,40)} in adipocyte differentiation, these results suggest that the iPLA₂ family of enzymes serves a critical role in the provision of at least some of the lipid second messengers required for the execution of adipocyte differentiation programs.

Knockdown of iPLA₂β or iPLA₂γ protein products inhibited the programmed expression of PPARγ and C/EBPα, known to be of decisive importance in commitment to the terminal phase of adipocyte differentiation. The block appears localized distal to the production of the early transcription factors C/EBPβ and C/EBPγ and prior to the production of the late transcriptional factors PPARγ and C/EBPα. These results suggest that lipids produced by iPLA₂ enzymes (or their downstream metabolites) modulate the transcription of PPARγ and C/EBPα. Moreover it seems likely that either iPLA₂β or iPLA₂γ (or both) provides the lipids or lipid precursors which serve to activate PPARγ (e.g. LPA and free fatty acids). Clonal expansion has generally been regarded as a prerequisite for terminal differentiation of cultured preadipocytes⁽²⁶⁾. The present students indicate iPLA₂β or iPLA₂γ siRNAs do not interfere with the reinitiation of cell cycling of growth-arrested 3T3-L1 preadipocytes induced by differentiation inducers. Since the expression of C/EBPβ and C/EBPδ mediated mitotic clonal expansion were not affected by iPLA₂β or iPLA₂γ siRNA pretreatment, these results localize the block in the programmed differentiation of 3T3-L1 cells into adipocytes as distal to these factors and proximal to the expression of PPARγ protein expression. Collectively, these results identify the involvement of the signaling pathways mediated by iPLA₂s in the commitment to the terminal phase of adipocyte differentiation.

Since it was first appreciated over a decade ago, many studies have attempted to identify the lipid or lipids responsible for PPARγ activation in adipocytes. Early studies demonstrated that a variety of eicosanoids could activate the PPARγ receptor⁽⁴¹⁻⁴³⁾. Recent studies have underscored the role of other lipids including LPA are also important^{(24,44)}. Thus, a potential role for fatty acids, eicosanoids and LPA, perhaps acting together, in activating PPARγ, is likely. Through adaptor or binding proteins which facilitate the delivery of lipid products to the PPARγ binding surface, it seems likely that both fatty acids, oxidized products and LPA act in concert. Thus, a variety of biologic, pharmacological and chemical techniques suggest that the endogenous activators of PPARγ⁽²⁴⁾ are likely fatty acids (and their downstream products) as well as LPA. Issues of concentration do not appear to be of concern with LPA as a PPARγ ligand since the concentrations of LPA necessary for PPARγ activation are similar to those found in biologic tissues and serum⁽²⁴⁾. Of course compartmentation and membrane surface effective mole fraction compositions are important issues which remain to be determitively addressed. Collectively, the results suggest a variety of lipids now known to be generated by iPLA₂β and iPLA₂γ in the adipocyte may modulate PPARγ activation (e.g. lysolipids, fatty acids, eicosanoids.)

The majority of evidence at this point suggests the importance of the intracellular production of LPC and its subsequent extracellular hydrolysis to LPA catalyzed by a secreted adipocyte lysophospholipase D, autotaxin. LPA was shown to be a positive regulatory mediator of adipogenesis by interacting preferentially with the LPA1 receptor (LPA1-R) after secretion^{(22,23)}. Moreover, expression of PPARγ can be autoactivated by its activation after ligand binding. Thus, cooperative interactions between PPARγ and C/EBPα are likely to be present as evidenced by the fact that ectopic expression of either transcription factor alone induces the expression of the other^{(37,45)}. This reciprocal gene activation also amplifies the effect of the PPARγ ligand mediated activation on the protein expression of PPARγ (feed forward activation).

Finally, it should be appreciated that multiple ligands may be important and that post-translational modification of PPARγ does occur. Differential phosphorylated forms of PPARγ may selectively bind to different lipids or perhaps have distinct downstream effectors depending on the nature of conformational shifts each ligand induces^{(46,47)}. Calcium-independent phospholipases A₂ may also regulate adipogenesis via productions of prostaglandins. PGF₂α is known to be synthesized by preadipocytes and its production is dramically decreased after induction of differentiation in 3T3-L1 cells⁽⁴⁸⁾. PGF₂α inhibits adipocyte differentiation via activation of MAP kinase and subsequent phosphorylation and inhibition of PPARγ^{(21,48)}. PGE₂ and PGI₂, the most abundantly produced PGs by mass, have differential effects on preadipocytes and adipocytes⁽¹⁹⁾. PGI₂ exclusively affects preadipocytes and induces adipogenesis by increasing intracellular cAMP and calcium while PGE₂ possesses an antilipolytic effect only in adipocytes⁽¹⁹⁾. Thus, it seems likely that iPLA₂s exert their proadipogenic effects by providing arachidonic acid used for the production of PGE₂ and PGI₂ in conjunction with the provision of LPC for subsequent hydrolysis by autotaxin, a fat cell secreted lysophospholipase D. It also seems likely that the production of the antiadipogenic PGF₂α, whose concentration decreases after induction of differentiation in 3T3-L1 cells, may be regulated by cPLA₂α whose protein product also dramatically decreases during the differentiation process. This invention describes the importance of iPLA₂β and iPLA₂γ and not cPLAα as previously believed to be the enzymic regulators of fat cell differentiation in mammalian cells.

Calcium homeostasis has been shown to play important, but complicated roles in adipocyte differentiation. Multiple reports have demonstrated an increase in intracellular calcium concentration ([Ca²⁺]ᵢ) during the early phase of 3T3-L1 and human preadipocyte differentiation inhibits hormone-induced adipogenesis^{(48,49)}. Additionally, increases in [Ca²⁺ ]ᵢ during the later phase of human preadipocyte differentiation induces TAG synthesis and the expression of specific adipocyte markers⁽⁴⁰⁾. The results from present study indicate that iPLA₂ may provide a calcium dependent switch in the regulation of adipocyte differentiation in response to the environmental or chemical stimuli such as adrenal corticotropinic hormone (ACTH)⁽⁵⁰⁾ and some PGs (e.g. PGF₂α)⁽⁴⁸⁾, which perturb intracellular calcium homeostasis. In this regard, important roles for iPLA₂β isoforms in cellular calcium homeostasis have recently been demonstrated^{(5,52)}. Previous work also identified the high affinity of iPLA₂β for ATP. ATP both stabilizes and activates iPLA₂β isoforms and thus is a positive regulator of iPLA₂β catalytic activities⁽⁵³⁻⁵⁵⁾. Accordingly, increased ATP levels resulting from increased glycolytic flux after insulin stimulation could be a positive regulator in adipogenic signaling pathways. Accordingly, the notion that iPLA₂s may be a sensor molecule which promotes the conversion of the excess chemical energy into lipid storage is consistent with the results of the present study.

Further evidence for a role of iPLA₂β and iPLA₂γ in adipocyte development and white adipocyte tissue maintenance was provided by tests utilizing genetically obese fa/fa rats. Western blot analysis demonstrated that the expression levels of iPLA₂β and iPLA₂γ were up-regulated in homozygous Zucker obese fa/fa rats relative to their congenic lean controls in WAT. This strong up-regulation of iPLA₂β and iPLA₂γ may contribute to the abnormal development and maintenance of WAT in these animals. Examination of iPLA₂β and iPLA₂γ expression levels in other obese animal models will further extend the observation.

Taken together, the present discovery is evidence of the disparate regulation of cPLA₂ and iPLA₂ classes of intracellular phospholipases during the hormone-induced differentiation of 3T3-L1 cells into adipocytes. The results identify the requirement of both iPLA₂β and iPLA₂γ in 3T3-L1 cell differentiation into adipocytes. It is now clear that increases in adipogenesis contribute to the development of obesity by increasing the number of mature adipocytes in multiple mammalian models. Thus, the present results provide a potential in vivo role for iPLA₂ in the regulation of obesity and the related pathophysiologic sequelae of the metabolic syndrome.

In an aspect, a method of TAG analysis and lipid analysis useful in this invention is carried out by using a method disclosed in U.S. patent application U.S. Ser. No. 10/606,601 filed Jun. 26, 2003, “Spectrometric Quantitation of Triglyceride Molecular Species” now publication 2004-0063108 published Apr. 1, 2004 which is incorporated herein by reference in its entirety. A level of expression greater or less than expression in an absence of the substance selected to be measured indicates and is determinant of activity in modulating iPLA₂ expression.

In a first embodiment, in regard to the method of analysis disclosed in U.S. Ser. No. 10/606,601, a method for the determination of TG individual (i.e. separate) molecular species in a composition of matter such as the above in a biological sample comprises subjecting the biological sample to lipid extraction to obtain a lipid extract and subjecting the lipid extract to electrospray ionization tandem mass spectrometry (ESI/MS/MS) providing TG molecular species composition as a useful output determination.

In an aspect, the inventive concept comprises analyzing a biological sample using electrospray ionization tandem mass spectrometry (ESI/MS/MS) and performing a two dimensional analysis with cross peak contour analysis on the output of the ESI/MS/MS to provide a fingerprint of triglyceride individual (i.e. separate) molecular species.

Briefly, the inventive methods present a novel two-dimensional approach/method which quantitates individual molecular species of triglycerides by two dimensional electrospray ionization mass spectroscopy with neutral loss scanning. This method is also useful for polar lipid analysis by ESI/MS using conditions as outlined in U.S. Ser. No. 10/606,601 (see above) and is protected by a provisional patent and be reference herein. This method provides a facile way to fingerprint each patient's (or biologic samples) triglyceride composition of matter (individual molecular species content) and lipid composition of matter directly from chloroform extracts of biologic samples. Through selective ionization and subsequent deconvolution of 2D intercept density contours of the pseudomolecular parent ions and their neutral loss products, the individual molecular species of triglycerides and phospholipids can be determined directly from chloroform extracts of biological material. This 2D (two dimensional) approach comprises a novel enhanced successful functional therapy model for the automated determination and global fingerprinting of each patient's serum or cellular triglyceride and phospholipid profile content thus providing the facile determination of detailed aspects of lipid metabolism underlying disease states and their response to diet, exercise or drug therapy.

In an aspect of this inventive method, tandem mass spectroscopic separation of specific lipid class-reagent ion pairs is used in conjunction with contour density deconvolution of cross peaks resulting from neutral losses of aliphatic chains to determine the individual triglyceride molecular species from a biological sample (blood, liver, muscle, feces, urine, tissue biopsy, or rat myocardium.).

In an aspect, a biological sample is processed in a tandem mass spectrometer, a first mass spectrometer set up in a tandem arrangement with another mass spectrometer. In that regard the biological sample can be considered as sorted and weighed in the first mass spectrometer, then broken into parts in an inter-mass spectrometer collision cell, and a part or parts of the biological sample are thereafter sorted and weighed in the second mass spectrometer thereby providing a mass spectrometric output readily and directly useable from the tandem mass spectrometer.

In an aspect, a pre-analysis separation comprises a separation of lipoproteins prior to lipid extraction. In an aspect, the pre-analysis separation comprises at least one operation or process which is useful to provide an enhanced biological sample to the electrospray ionization tandem mass spectrometry (ESI/MS/MS). In an aspect, a pre-analysis separation is performed on a biological sample and two compositions are prepared accordingly from the biological sample. In an aspect one composition comprises high density lipoproteins and another composition comprises low density lipoproteins and variants thereof comprised of intermediate densities which can, if necessary, be resolved by chromatographic or other density techniques.

Generally, a biological sample taken is representative of the subject from which or of which the sample is taken so that an analysis of the sample is representative of the subject preferably a living subject such as living cells such as an animal. In an aspect a representative number of samples are taken and analyzed of a subject such that a recognized and accepted statistical analysis indicates that the analytic results are statistically valid. Typically the composition is aqueous based and contains proteinaceous matter along with triglycerides. For example, a human blood sample is sometimes used. Through use of this inventive method, a plasma sample can be analyzed and appropriate information from the plasma can be extracted in a few minutes. Alternatively, information can be taken from the cells in the blood as well.

In an aspect, serum is utilized as a biological sample. After whole blood is removed from a human body and the blood clots outside the body, blood cells and some of the proteins become solid leaving a residual liquid which is serum.

In an aspect a control sample is employed in the analysis.

In an aspect, the biological sample or a representative aliquot or portion thereof is subjected to lipid extraction to obtain a lipid extract suitable for ESI/MS/MS. In an aspect lipids are extracted from the sample which in an aspect contains a tissue matrix. Non-lipid contaminants should be removed from the lipid extract.

In one aspect lipid extraction is carried by the known lipid extraction process of Folch as well as by the known lipid extraction process of Bligh and Dyer. These useful lipid extraction process are described in Christie, W.W. Preparation of lipid extracts from tissues. In: Advances in Lipid Methodology-Two, pp. 195-213 (1993) (edited by W. W. Christie, Oily Press, Dundee) EXTRACTION OF LIPIDS FROM SAMPLES William W. Christie The Scottish Crop Research Institute, Invergowrie, Dundee DD2 5DA, Scotland all of which are incorporated herein in their entirety by reference. The useful Folch extraction process is reported at Folch et al. (1957) J Biol Chem 226, 497-509 which is incorporated herein in its entirety by reference.

Generally, lipid extraction is carried out very soon in time on the tissue matrix or immediately after removal (harvest) of tissues (tissue matrix) from humanely sacrificed organisms which have been living (carried out using and following acceptable animal welfare protocols). Alternatively, tissues are stored in such a way that they are conservatively preserved for future use. In an aspect, a lipid extract is provided and used to produce ionized atoms and molecules in the inventive analytical method as feed to the ESI mass spectrometer in our novel analysis method.

In an aspect a chloroform lipid extract is employed as a lipid extract composition fed to the ESI mass spectrometer. The effluent from the ESI is fed to the tandem mass spectrometer (i.e. from the exit of the ESI).

In an aspect, a Freezer Mill 6800 from Fisher Bioblock Scientific is used to finely pulverize soft or hard harvested tissues of a biological sample in one or two minutes in liquid nitrogen to render the tissue sufficiently pliable and porous for lipid extraction. Alternatively, the pulverization of the harvested tissue is carried out by subjecting the harvested tissue to hand directed mashing and pulverization using a hand directed stainless-steel mortar and pestle. In a further aspect, an enzymatic digestion is carried out on the harvested tissue which is harvested from a preserved cadaver.

In an aspect, lipids are contained in the lipid extract following the lipid extraction. Generally the extraction is a suitable liquid/liquid or liquid/solid extraction whereby the TG are contained in the extract. In an aspect the extractant has sufficient solvating capability power and solvating capacity so as to solvate a substantial portion of the TG therein or substantially all of the TG present in the biological sample and is contained in the lipid extract.

In an aspect, chloroform is employed as an extractant to produce a useful lipid extract. Other useful extractants include but are not limited to those extractants which have a solvating power, capability and efficiency substantially that of chloroform with regard to the TG molecular species.

The inventive process creates charged forms of very high molecular weight TG molecules obtained via lipid extraction of a biological sample as a part of the process of detecting and analyzing biological samples containing TG.

In an aspect, in order to detect for and analyze ionized atoms and molecules such as TG molecular species in a biological sample, the lipid extract of that biological sample is used to produce ionized atoms and molecules by an ionization method such as electrospray ionization (ESI). As used herein, the term ESI includes both conventional and pneumatically-assisted electrospray mass spectrometry.

In use, the inventive procedure operates by producing droplets of a sample composition by pneumatic nebulization which compresses and forces a biological sample composition containing TG such as an analyte containing TG into a proximal end of a mechanical means housing or holding a fine sized orifice such as a needle or capillary exiting at the distal end of the orifice at which there is applied a sufficient potential. Generally the orifice is a very small bore full length orifice having an internal average diameter or bore in the range from about 0.2 to about 0.5 mm.

In an aspect, formation of a suitable spray is a critical operating parameter in ESI. Suitable solvent removable filters may be used to remove undesired solvents in the biological sample composition prior to being fed to the ESI. Generally high concentrations of electrolytes are avoided in samples fed to ESI.

The composition of materials of the means housing or holding the orifice and the orifice are compatible with the compositions of the biological sample to be processed through the orifice. Metallic and composition plastic compositions may be employed. In an aspect the orifice is a capillary or has a conical or capillary shape. In another aspect the orifice is cone shaped with the exterior converging from the proximate end to the distal end.

In an aspect, the biologic sample is forced through the orifice by application of air pressure to the sample at the proximate end of the orifice or the sample is forced through the orifice or capillary by the application of vacuum at the distal end of the orifice. The net result is that ions are suitably formed at atmospheric pressure and progress through the cone shaped orifice. In an aspect the orifice is a first vacuum stage and the ions undergo free jet expansion. A collector at the distal end of the orifice collects the ions and guides the ions to a tandem mass spectrometer (MS/MS).

The construction of a suitable vector can be achieved by any of the methods well-known in the art for the insertion of exogenous DNA into a vector. See Sambrook et al., 1989, Molecular Cloning, A Laboratory Manual Cold Spring Harbor Press, N.Y.; Rosenberg et al., Science 242:1575-1578 (1988); Wolff et al., Proc Natl Acad Sci USA 86:9011-9014 (1989). For Systemic administration with cationic liposomes, and administration in situ with viral vectors, see Caplen et al., Nature Med., 1:39-46 (1995); Zhu et al., Science 261:209-211 (1993); Berkner et al., Biotechniques, 6:616-629 (1988); Trapnell et al., Advanced Drug Delivery Rev., 12:185-199 (1993); Hodgson et al., BioTechnology 13:222 (1995).

### REFERENCES

1. Friedman, J. M. (2000) Nature 404, 632-634
2. Spiegelman, B. M., and Flier, J. S. (2001) Cell 104, 531-543
3. Gregoire, F. M., Smas, C. M., and Sul, H. S. (1998) Physiol. Rev. 78, 783-809
4. Friedman, J. M., and Halaas, J. L. (1998) Nature 395, 763-770
5. Steppan, C. M., Bailey, S. T., Bhat, S., Brown, E. J., Banerjee, R. R., Wright, C. M., Patel, H. R., Ahima, R. S., and Lazar, M. A. (2001) Nature 409, 307-312
6. Moustaid, N., and Sul, H. S. (1991) J. Biol. Chem. 266, 18550-18554
7. MacDougald, O. A., and Lane, M. D. (1995) Annu. Rev. Biochem. 64, 345-373
8. Xue, J.-C., Schwarz, B. J., Chawla, A., and Lazar, M. A. (1996) Mol. Cell. Biol. 16, 1567-1575
9. Prusty, D., Park, B.-H., Davis, K. E., and Farmer, S. R. (2002) J. Biol. Chem. 277, 46226-46232
10. Mandrup, S., and Lane, M. D. (1997) J. Biol. Chem. 272, 5367-5370
11. MacDougald, O. A., and Mandrup, S. (2002) Trends Endocrinol. Metab. 13, 5-11
12. Cornelius, P., MacDougald. O. A., and Lane, M. D. (1994) Annu. Rev. Nutri. 14, 99-129
13. Rosen, E. D., and Spiegelman, B. M. (2001) J. Biol. Chem. 276, 37731-37734
14. Gao, G., and Serrero, G. (1990) J. Biol. Chem. 265, 2431-2434
15. Gross, R. W. (1992) Trends Cardiovasc. Med. 2, 115-121
16. Gross, R. W. (1995) J Lipid Mediat. Cell Signal. 12, 131-137
17. Miyake, R., and Gross, R. W. (1992) Biochim. Biophys. Acta 1165, 167-176
18. Kudo, I., and Murakami, M. (2002) Prostaglandins Other Lipid Mediat. 68/69, 3-58
19. Vassaux, G., Gaillard, D., Darimont, C., Ailhaud, G., and Negrel, R. (1992) Endocrinology 131, 2393-2398
20. Vassaux, G., Gaillard, D., Ailhaud, G., and Negrel, R. (1992) J. Biol. Chem. 267, 11092-11097
21. Reginato, M. J., Krakow, S. L., Bailey, S. T., and Lazar, M. A. (1998) J. Biol. Chem. 273, 1855-1858
22. Ferry, G., Tellier, E., Try, A., Gres, S., Naime, I., Simon, M. F., Rodriguez, M., Boucher, J., Tack, I., Gesta, S., Chomarat, P., Dieu, M., Raes, M., Galizzi, J. P., Valet, P., Boutin, J. A., and Saulnier-Blache, J. S. (2003) J. Biol. Chem. 278, 18162-18169
23. Hooks, S. B., Santos, W. L., Im, D.-S., Heise, C. E., Macdonald, T. L., and Lynch, K. R. (2001) J. Biol. Chem. 276, 4611-4621
24. McIntyre, T. M., Pontsler, A. V., Silva, A. R., St. Hilaire, A., Xu, Y., Hinshaw, J. C., Zimmerman, G. A., Hama, K., Aoki, J., Arai, H., and Prestwich, G. D. (2003) Proc. Natl. Acad. Sci. USA 100, 131-136
25. Tang, Q. Q., Otto, T. C., and Lane, M. D. (2003) Proc. Natl. Acad. Sci. USA 100, 850-855
26. Tang, Q. Q., Otto, T. C., and Lane, M. D. (2003) Proc. Natl. Acad. Sci. USA 100, 44-49
27. Altiok, S., Xu, M., and Spiegelman, B. M. (1997) Genes Dev. 11, 1987-1998
28. Wang, H., lakova, P., Wilde, M., Welm, A., Goode, T., Roesler, W. J., and Timchenko, N. A. (2001) Mol. Cell 8, 817-828
29. Mancuso, D. J., Jenkins, C. M., and Gross, R. W. (2000) J. Biol. Chem. 275, 9937-9945
30. Frost, S. C., and Lane, M. D. (1985) J. Biol. Chem. 260, 2646-2652
31. Gomez, F. E., Miyazaki, M., Kim, Y. C., Marwah, P., Lardy, H. A., Ntambi, J. M., and Fox, B. G. (2002) Biochemistry 41, 5473-5482
32. Jenkins, C. M., Han, X., Mancuso, D. J., and Gross, R. W. (2002) J. Biol. Chem. 277, 32807-32814
33. Bligh, E. G., and Dyer, W. J. (1959) Can. J. Biochem. Physiol. 37, 911-917
34. Han, X., and Gross, R. W. (2001) Anal. Biochem. 295, 88-100
35. Rosen, E. D., Sarraf, P., Troy, A. E., Bradwin, G., Moore, K., Milstone, D. S., Spiegelman, B. M., and Mortensen, R. M. (1999) Mol. Cell 4, 611-617
36. Wu, Z., Rosen, E. D., Brun, R., Hauser, S., Adelmant, G., Troy, A. E., MeKeon, C., Darlington, G. J., and Spiegelman, B. M. (1999) Mol. Cell 3, 151-158
37. Schwarz, E. J., Reginato, M. J., Shao, D., Krakow, S. L., and Lazar, M. A. (1997) Mol. Cell. Biol. 17, 1552-1561
38. Morrison, R. F., and Fanner, S. R. (2000) J Nutr. 130, 3116S-3121S
39. Descombes, P., and Schibler, U. (1991) Cell 67, 569-579
40. Shi, H., Halvorsen, Y. D., Ellis, P. N., Wilkison, W. O., and Zemel, M. B. (2000) Physical Genomics 3, 75-82
41. Huang, J. T., Welch, J. S., Ricote, M., Binder, C. J., Willson, T. M., Kelly, C., Witztum, J. L., Funk, C. D., Conrad, D., and Glass, C. K. (1999) Nature 400, 378-382
42. Forman, B. M., Tontonoz, P., Chen, J., Brun, R. P., Spiegelman, B. M., and Evans, R. M. (1995) Cell 83, 803-812
43. Nosjean, O., and Boutin, J. A. (2002) Cell. Signal. 14, 573-5 83
44. Bell-Parikh, L. C., Ide, T., Lawson, J. A., McNamara, P., Reilly, M., and FitzGerald, G. A. (2003) J. Clin. Invest. 112, 945-955
45. Tontonoz, P., Hu, E., and Spiegelman, B. M. (1994) Cell 79, 1147-1156
46. Shao, D., Rangwala, S. M., Bailey, S. T., Krakow, S. L., Reginato, M. J., and Lazar, M. A. (1998) Nature 396, 377-380
47. Lazennec, G., Canaple, L., Saugy, D., and Wahli, W. (2000) Mol. Endocrinol. 14, 1962-1975
48. Miller, C. W., Casimir, D. A., and Ntambi, J. M. (1996) Endocrinology 137, 5641-5650
49. Ntambi, J. M., and Takova, T. (1996) Differentiation 60, 151-158
50. Izawa, T., Mochizuki, T., Komabayashi, T., Suda, K., and Tsuboi, M. (1994) Am. J. Physiol. 266, E418-426
51. Smani, T., Zakharov, S. I., Leno, E., Csutora, P., Trepakova, E. S., and Bolotina, V. M. (2003) J. Biol. Chem. 278, 11909-11915
52. Hichami, A., Joshi, B., Simonin, A. M., and Khan, N. A. (2002) Eur. J. Biochem. 269, 5557-5563
53. Hazen, S. L., and Gross, R. W. (1991) Biochem J 280, 581-587
54. Hazen, S. L., and Gross, R. W. (1991) J. Biol. Chem. 266, 14526-14534
55. Ramanadham, S., Wolf, M. J., Ma, Z., Li, B., Wang, J., Gross, R. W., and Turk, J. (1996) Biochemistry 35, 5464-5471

While the invention has been described in terms of various specific embodiments, those skilled in the art will recognize that the invention can be practiced with modification within the spirit and scope of the claims.

1. A method for controlling adipocyte differentiation using molecular biologic inhibition in at least one effective against the activity of at least one target selected from iPLA₂β and iPLA₂γ which comprises culturing cells, competently transfecting the cells with an effective amount siRNA directed against iPLA₂β or siRNA directed against iPLA₂γ prior to induction of differentiation and observing if down regulation of iPLA₂β or iPLA₂γ inhibits or controls adipocyte chemistry (lipid or protein markers) during differentiation.

2. A method in accordance with claim 1 wherein the cells comprise 3T3 L1 cells and concluding that adipocyte differentiation and hypertrophy are affected by siRNA directed against iPLA₂β or iPLA₂γ.

3. A method for controlling adipocyte differentiation using pharmacologic inhibition effective against the activity of at least one of iPLA₂β and/or iPLA₂γ which comprises culturing cells, administering a compound thereto prior to induction to differentiation and observing if down regulation of iPLA₂β or iPLA₂γ inhibits or controls adipocyte differentiation and concluding that the compound is effective if triglycerides do not accumulate as determined by ESI/MS and the cells do not differentiate.

4. A method in accordance with claim 3 wherein cells comprise 3T3-L1 cells and determining that down regulation of iPLA₂β or iPLA₂γ inhibits adipocyte differentiation if cells do not accumulate fat or differentiate and determining that the compound is an inhibitor of said processes.

5. A method of modulating the amount of fat in a living mammal having differentiable preadipocytes which comprises transfecting cells with siRNA directed against iPLA₂β or siRNA directed against iPLA₂γ prior to induction of preadipocytes to differentiation, growing the cells and examining cells for alterations in lipid metabolism, or alterations in profiles typically associated with adipocyte differentiation (e.g. triglyceride mass) and determining that the siRNA is effective if the cells remain in a pre-adipogenic state (the cells fail to differentiate and accumulate triglycerides).

6. A method in accordance with claim 4 wherein the amount of fat is decreased.

7. A method in accordance with claim 4 wherein the cells are examined for alterations in lipid metabolism.

8. A method in accordance with claim 4 wherein the cells are examined for alterations in profiles associated with adipocyte differentiation (e.g. triglyceride mass).

9. A method in accordance with claim 5 wherein the cells are examined for alterations in triglyceride mass.

10. A method in accordance with claim 8 wherein the triglyceride mass is obtained by ESI/MS/MS and the amount of fat is decreased.

11. A method of controlling the rate of differentiation of preadipocytes to adipocytes in a living mammal (e.g. murine or human) having differentiable preadipocytes and expressible iPLA₂β or iPLA₂γ which comprises administering a pharmacologically effective amount of an inhibitor to the activity of iPLA₂β or the activity of iPLA₂γ to the living animal.

12. A method in accordance with claim 10 wherein the inhibitor is administered to the living animal which is a human.

13. A method in accordance with claim 10 wherein the inhibitor is a chemical compound which is administered to a living nonhuman mammal.

14. A method to screen for endogenous regulators of iPLA₂β or iPLA₂γ wherein the effects on fat cell differentiation or lipid alterations are attenuated by administration of a chemical to a living mammal expressing iPLA₂β or iPLA₂γ and determining that the chemical is a regulator if the expression or activity of iPLA₂β or iPLA₂γ is altered.

15. A method in accordance with claim 13 wherein the living mammal is a human and the effects are measured by using ESI/MS/MS.

16. A method of modulating the amount of fat in a living mammal (e.g. human or murine) having differentiable preadipocytes which comprises administering a compound directed against of iPLA₂β or directed against iPLA₂γ (or both) prior to induction of preadipocytes to differentiation whereby the amount of fat is increased or decreased in the living mammal.

17. A method in accordance with claim 16 wherein the living mammal is a human and amount of fat is decreased.

18. A method in accordance with claim 16 wherein the living mammal is a murine.

19. A method in accordance with claim 18 wherein the murine is wild or transgenic mouse.

20. A method for identifying a molecular biologic inhibition by siRNA knockdown, which comprises transfecting cells (e.g. murine or human) with siRNA directed against of iPLA₂β or siRNA directed against iPLA₂γ prior to induction of preadipocytes to differentiation, growing the cultured cells and measuring protein expression or lipid levels, comparing the amount and types of proteins and lipids with control cells and determining that the siRNA knockdown or pharmacologic inhibitor was effective if the protein expression and/or lipid levels do not change from baseline values.

21. A method in accordance with claim 20 wherein the inhibitor is determined to be effective if the protein expression did not change from a baseline value.

22. A method in accordance with claim 20 wherein the inhibitor is a chemical compound and the inhibitor is determined to be effective if the lipid levels did not change from baseline values.

23. A method in accordance with claim 22 wherein the lipid levels are measured by using ESI/MS/MS.

24. A method for identifying a molecular biologic inhibition by siRNA knockdown, which comprises transfecting 3T3-L1 cultured cells with pharmacologic inhibition directed against iPLA₂β or siRNA directed against iPLA₂γ (or both) prior to induction of preadipocytes to differentiation and measuring protein expression or lipid levels to determine a baseline value, comparing the amount and types of proteins and lipids with control cells and determining that the siRNA knockdown or pharmacologic inhibitor was effective if the levels do not change from a baseline value.

25. A method in accordance with claim 24 wherein the cells comprises 3T3-L1cells.

26. A method in accordance with claim 24 wherein the inhibitor was siRNA knockdown.

27. A method in accordance with claim 24 wherein the inhibitor was pharmacologic.

28. A method in accordance with claim 24 wherein the amounts and types of protein were compared using ESI/MS/MS.

29. A method for identifying (determining) a pharmacological inhibitor of fat in living tissue which comprises administering a compound to a living tissue (e.g. murine or human) having differential preadipocytes and expressible iPLA₂β or iPLA₂γ, inducing differentiation and measuring the change in activity of the expressible iPLA₂β or iPLA₂γ, and determining that if the compound is a pharmacological inhibitor of fat when lipid or protein expression levels or flux are altered.

30. A method in accordance with claim 29 wherein the tissue is murine.

31. A method in accordance with claim 29 wherein the tissue is human.

32. A functional animal model useful for identifying a pharmacological inhibitor of fat in the model which comprises a target tissue having differentiable preadipocytes and expressible iPLA₂β or iPLA₂γ therewith inducing differentiation, and measurable altered serum and fat and/or lipids or proteins and determining the effects of these altered serum lipids or tissue lipids have on the end-organ metabolic flux.

33. A model in accordance with claim 32 wherein the model is a living murine.

34. A method for identifying an agonist exhibiting molecular biologic inhibition which is effective against the activity of at least one of iPLA₂β or iPLA₂γ which comprises culturing cells and transfecting them with siRNA directed against of iPLA₂β or iPLA₂γ (or both), or pharmacologic inhibition prior to induction of differentiation and observing for down regulation of iPLA₂β or iPLA₂γ (or both) if adipocyte differentiation is altered and identify the agonist by conducting tests with increasing concentrations of agonist to produce inhibition of fat cell differentiation.

35. A method in accordance with claim 33 wherein the cells comprise 3T3-L1 cells.

36. A method to identify associated regulatory elements which modulate iPLA₂β or iPLA₂γ activity or act in concert with iPLA₂β or iPLA₂γ effects on fat cell differentiation which comprises at least one of culturing 3T3-L1 cells and treating them with an effective amount of negative control siRNA, siRNA directed against iPLA₂β or siRNA directed against iPLA₂γ prior to induction to differentiation and observing if down regulation of iPLA₂β or iPLA₂γ inhibits adipocyte differentiation and treating 3T3-L1 cultured cells, administering a compound thereto prior to induction of preadipocytes to differentiation and determining effect of the addition of the compound on a regulatory eluent.

37. A method in accordance with claim 36 wherein the compound is chemical compound.

38. A method in accordance with claim 36 wherein the siRNA is directed against iPLA₂β.

39. A method in accordance with claim 36 wherein the siRNA is directed against iPLA₂γ.

40. A method in accordance with claim 36 wherein a regulator element is identified as a sequence of DNA or RNA acting as a molecular switch if the effect is control of iPLA₂β or iPLA₂γ expression.

41. A screening tool which comprises a living tissue having differentiable preadipocytes and expressible iPLA₂β or iPLA₂γ, and an administration method of administering a silencing gene thereto or a pharmacological inhibitor effecting thereto and means for determining any change in metabolics of the system through analysis of a biological sample.

42. A tool in accordance with claim 40 wherein the sample is analyzed by ESI/MS/MS for fat content or lipid metabolic flux.

43. A tool in accordance with claim 41 wherein the sample is analyzed for fat content by ESI/MS/MS.

44. A tool in accordance with claim 41 wherein the sample is analyzed for lipid metabolic flux by ESI/MS/MS.
